SEQUENCE LISTING (6 sequences)

SEQ ID NO: 1 - DNA, 28 bp, Artificial sequence
  Synthetic primer
aaacaaccccaagctaactgcgacaagg

SEQ ID NO: 2 - DNA, 27 bp, Artificial sequence
  Synthetic primer
ccagaaccatgcccggattctgacttc

SEQ ID NO: 3 - DNA, 25 bp, Artificial sequence
  Synthetic primer
ccaagtgcccagcggggctgctaaa

SEQ ID NO: 4 - DNA, 21 bp, Artificial sequence
  Synthetic primer
cactatcccactgacccttca

SEQ ID NO: 5 - DNA, 20 bp, Artificial sequence
  Synthetic primer
agaaggtgagggacctccag

SEQ ID NO: 6 - DNA, 20 bp, Artificial sequence
  Synthetic primer
cacagggtaggcatgtagca

(19) United States
(12) Patent Application Publication
(10) Pub. No.: US 2022/0053740 A1
(43) Pub. Date: Feb. 24, 2022

(54) A GENETIC MOUSE MODEL OF AUTOIMMUNE ADVERSE EVENTS AND IMMUNE CHECKPOINT BLOCKADE THERAPY

(71) Applicant: Board of Regents, The University of Texas System, Austin, TX (US)

(72) Inventors: Spencer WEI, Houston, TX (US); James ALLISON, Houston, TX (US)

(73) Assignee: Board of Regents, The University of Texas System, Austin, TX (US)

(21) Appl. No.: 17/274,890

(22) PCT Filed: Sep. 11, 2019
(86) PCT No.: PCT/US2019/050551

§ 371 Date: Mar. 10, 2021

Related U.S. Application Data
(60) Provisional application No. 62/729,965, filed on Sep. 11, 2018.

(51) Int. Cl. A01K 67/027; A61K 49/00

(52) U.S. Cl. CPC A01K 67/0276; A61K 49/0008; A01K 2267/0325; A01K 2217/077; A01K 2227/105; A01K 2267/0387; A01K 2217/075

(57) ABSTRACT

Provided herein are mice that are heterozygous knock outs for Ctla4 and homozygous knockouts for Pdcd1 (Ctla4^{+/-} Pdcd1^{-/-} mice), which may suffer from autoimmunity, including myocarditis and insulin-dependent diabetes mellitus. Also provided are methods of using such mice to screen for therapeutic agents that mitigate immune-related adverse events.

This application claims the benefit of U.S. Provisional Patent Application No. 62/729,965, filed Sep. 11, 2018, the entirety of which is incorporated herein by reference.

### BACKGROUND

### 1. Field

The present invention relates generally to the field of immunology. More particularly, it concerns an animal model for the autoimmune disease etiologies that arise as a result of immune checkpoint blockade therapies as well as methods of using the animal model to screen for agents that mitigate said disease etiologies.

### 2. Description of Related Art

T cell activation is an exquisitely regulated biological process that enables the generation of rapid and highly sensitive responses to foreign antigens while maintaining the ability to distinguish self from non-self and prevent autoimmunity. A key concept underlying this remarkable process is that multiple distinct signals are required to fully activate, or prime, naïve T cells. These cues include cognate antigen recognition by the T-cell receptor (TCR) (Signal 1) and CD28 positive co-stimulation (Signal 2). Because CD28 positive co-stimulation is provided by professional antigen presenting cells, this enforces a cell extrinsic requirement for robust T cell activation. The next key step is negative co-stimulation, which is a feedback regulatory mechanism that attenuates T cell activation through inhibition of Signals 1 and 2. CTLA4 and PD-1 are principal negative costimulatory molecules that attenuate T cell activation through distinct molecular mechanisms. While CTLA4 attenuates T cell activation via competitive inhibition of CD28 positive co-stimulation, PD-1 primarily acts to inhibit proximal T-cell receptor (TCR) signaling via the phosphatase SHP2 (Chemnitz et al., 2004; Krummel & Allison, 1996; Parry et al., 2005; Walunas et al., 1996). Recent evidence suggests that PD-1 also leads to inhibition of CD28 positive costimulation (Hui et al., 2017) and relatedly, that CD28 signaling is required for effective responses to PD-1 blockade (Kamphorst et al., 2017). This suggests that attenuation of CD28 may be a shared mechanism of PD-1 and CTLA4 mediated T cell regulation.

T cell activation is generally thought to be governed by a threshold model, in which TCR and costimulatory signals must meet a minimum level to trigger activation. It is unknown whether PD-1 and CTLA4 negative co-stimulation lead to convergent functional regulation or alternatively, whether they exert distinct regulatory pressures to define the activation threshold. Relatedly, the relative functional contribution of the specific mechanisms of CTLA4 and PD-1 to T cell attenuation remains unclear. It is possible that these distinct mechanisms converge at the molecular, cellular, and/or tissue level. For example, at the molecular level, CTLA4 and PD-1 may co-regulate T cell signaling in a cell intrinsic manner through inhibition of CD28. At the cellular level, CTLA4 and PD-1 attenuate T cells with distinct kinetics with respect to activation and it is unclear whether and how such temporally separated regulation is integrated. Thus, a critical open fundamental question is whether the distinct regulatory mechanisms of CTLA4 and PD-1 negative co-stimulation functionally interact.

Anti-CTLA4 and anti-PD-1 therapies are effective in multiple tumor types advanced melanoma and renal cell carcinoma. However, immune checkpoint blockade therapy can induce serious immune-related adverse events as well as bona fide autoimmunity, such as myocarditis and type I diabetes in rare instances. There are currently no animal models that faithfully recapitulate the adverse events associated with checkpoint blockade therapy. Treatment of mice with checkpoint blockade antibodies (i.e. anti-CTLA4, anti-PD-1) does not lead to significant pathologies and does not faithfully recapitulate the range, severity, and type of immune related adverse events seen in human patients. In particular, these models do not recapitulate the rare autoimmune diseases that are associated with checkpoint blockade. As such, animal models that recapitulate the adverse events associated with checkpoint blockade therapy are needed.

### SUMMARY

Provided herein is an animal model that recapitulates autoimmunity induced by checkpoint blockade in human patients. In one embodiment, a mouse is provided whose genome comprises: (i) a heterozygous loss-of-function of a Ctla4 gene and (ii) a homozygous loss-of-function of a Pdcd1 gene. In one aspect, the mouse has a C57BL/6J genetic background. In some aspects, the mouse is a female mouse. In some aspects, the mouse is a male mouse.

In some aspects, the heterozygous loss-of-function allele of a Ctla4 gene is further defined as a heterozygous insertion of a neomycin resistance cassette into exon 3 of the Ctla4 gene. In some aspects, the homozygous loss-of-function allele of the Pdcd1 gene is further defined as a homozygous deletion of exons 2 and 3 of the Pdcd1 gene. In one aspect, the mouse is a Ctla4^{tm1All}Pdcd1^{tm1.1Shr} mouse.

In some aspects, the mouse suffers from autoimmunity. In certain aspects, the autoimmunity is cardiac autoimmunity or pancreatic autoimmunity. In one aspect, the cardiac autoimmunity is myocarditis. In certain aspects, the myocarditis is fulminant myocarditis. In one aspect, the pancreatic autoimmunity is insulin-dependent diabetes mellitus or lymphocytic pancreatitis. In some aspects, the pancreatic autoimmunity results in pancreatic exocrine destruction or pancreatic islet destruction. In some aspects, the autoimmunity is lymphocytic myocarditis, endarteritis, pancreatic exocrine destruction, pulmonary vasculitis, adipose tissue atrophy (both white and brown), hepatic inflammation, atrophy of female reproductive organs, gastrointestinal tract inflammation, synovitis, or lymphocytic infiltration of the kidney, salivary gland, lacrimal gland, or stomach.

In one embodiment, a cell isolated from a mouse of any of the present embodiments is provided. In some aspects, the cell is an immune cell. In some aspects, the cell is a T cell.

In one embodiment, methods are provided for screening at least one candidate agent in a mouse of the present embodiments, the methods comprising administering one or more candidate agent to the mouse. In some aspects, the methods further comprise screening the at least one candidate agent in a mouse comprising (i) a homozygous wild-type Ctla4 gene and (ii) a homozygous loss-of-function of a Pdcd1 gene; a mouse comprising (i) a homozygous wild-type Ctla4 gene and (ii) a homozygous wild-type Pdcd1 gene; a mouse comprising (i) a homozygous wild-type Ctla4 gene and (ii) a heterozygous loss-of-function allele of a Pdcd1 gene; a mouse comprising (i) a heterozygous loss-of-function allele of a Ctla4 gene and (ii) a homozygous wild-type Pdcd1 gene; and/or a mouse comprising (i) a heterozygous loss-of-function allele of a Ctla4 gene and (ii) a heterozygous loss-of-function of a Pdcd1 gene; and/or a mouse comprising (i) a heterozygous loss-of-function allele of a Ctla4 gene and (ii) a homozygous loss-of-function allele of a Pdcd1 gene.

In some aspects, the at least one candidate therapeutic agent is screened for its ability to mitigate an immune-related adverse event or immune-related condition. In some aspects, mitigating an immune-related adverse event or immune-related condition is further defined as preventing the development of the immune-related adverse event or immune-related condition. In some aspects, mitigating an immune-related adverse event or immune-related condition is further defined as decreasing the severity of the immune-related adverse event or immune-related condition. In some aspects, mitigating an immune-related adverse event or immune-related condition is further defined as mitigating the mortality resulting from Ctla4 haploinsufficiency. In some aspects, mitigating an immune-related adverse event or immune-related condition is further defined as mitigating a systemic immune-related adverse event or immune-related condition. In some aspects, mitigating an autoimmunity is further defined as mitigating organ- or tissue-specific autoimmunity. In some aspects, mitigating an immune-related adverse event or immune-related condition is further defined as decreasing the severity of the immune-related adverse event or immune-related condition. In some aspects, mitigating an immune-related adverse event or immune-related condition is further defined as decreasing the frequency at which the immune-related adverse event or immune-related condition manifests in the population of mice. In some aspects, mitigating an immune-related adverse event or immune-related condition is further defined as slowing the development, or time to onset, of the immune-related adverse event or immune-related condition. In some aspects, screening a candidate therapeutic agent is defined as testing the efficacy of the candidate therapeutic agent.

In some aspects, the immune-related adverse event or immune-related condition is inflammation, such as, for example, acute inflammation or chronic inflammation. In some aspects, the immune-related adverse event or immune-related condition is autoimmunity or an autoimmune condition. In some aspects, the immune-related adverse event or immune-related condition comprises an immune-related adverse event or immune-related condition that mimics an immune-related adverse event or an autoimmunity induced by a checkpoint blockade therapy in humans. In certain aspects, the immune-related adverse event or immune-related condition is cardiac autoimmunity or pancreatic autoimmunity. In one aspect, the cardiac autoimmunity is myocarditis, such as, for example, fulminant myocarditis. In one aspect, the pancreatic autoimmunity is insulin-dependent diabetes mellitus.

In some aspects, the candidate agent is a CTLA4-immunoglobulin fusion protein (e.g., abatacept or a murine version thereof), a steroid, an agent that depletes a specific population of immune cells (e.g., anti-CD4 antibody to deplete CD4 T cells or anti-CD20 monoclonal antibody (e.g., rituximab) to deplete B cells), a cytokine modulating agent (e.g., toclizumab or a murine version thereof), or an immunosuppressive agent. In some aspects, the candidate agent is an anti-cancer therapy (e.g., chemotherapy, radiation, surgery, kinase inhibitors, immunotherapies, anti-TIM3, anti-OX40, oncolytic viruses, bispecific antibodies) and the method is screening for additional adverse events that occur in mice suffering from autoimmunity that mimics an immune-related adverse event. The screening may identify therapeutic agents, that when combined with immune checkpoint blockade, have an unfavorable risk profile for the development of autoimmunity or immune-related adverse events. In some aspects, the candidate agent is a pathogen (e.g., commensal or infectious), stress, an injury, and/or a diet. In some aspects, the candidate agent is a tumor cell, such as a syngeneic tumor cell (e.g., B16 melanoma, MC38 colon carcinoma, Lewis lung carcinoma). The tumor cell may be engrafted into the mouse and the effect of the resulting tumor on the immune-related adverse events may be characterized. Tumor properties tested may include total tumor burden, tumor lysis resulting from therapy, release of tumor-associated antigens (e.g., injection of irradiated tumor cells as a tumor immunization), or specific properties of the tumor (e.g., specific mutations or activity of specific genes).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C. Genetic interaction between Ctla4 and Pdcd1 reveals lethal haploinsufficiency phenotypes. (FIG. 1A) Kaplan-Meier survival curve of transgenic C57BL6/J mice harboring Ctla4 and Pdcd1 knockout alleles (n=138 total mice with n=5 Ctla4^{+/+} Pdcd1^{+/+}, n=6 Ctla4^{+/+} Pdcd1^{+/-}, n=32 Ctla4^{+/+} Pdcd1^{-/-}, n=10 Ctla4^{+/-} Pdcd1^{+/+}, n=50 Ctla4^{+/-} Pdcd1^{+/-}, n=14 Ctla4^{+/-} Pdcd1^{-/-}, n=14 Ctla4^{-/-} Pdcd1^{+/+}, and n=7 Ctla4^{-/-} Pdcd1^{+/-} mice). Mice were derived from an intercross of Ctla4^{+/-} Pdcd1^{+/-} mice in which Pdcd1 and Ctla4 loss of function alleles are in trans. Individual mice were censored if used for breeding or alive at the time of data analysis. Death events were defined as mice found dead or identified by veterinary staff as requiring euthanasia. (FIG. 1B) Kaplan-Meier survival curve of Ctla4^{+/-} Pdcd1^{-/-} (n=102) and littermate Ctla4^{+/+} Pdcd1^{-/-} (n=106) mice derived from a breeding cross of male Ctla4^{+/-} Pdcd1^{-/-} and female Ctla4^{+/+} Pdcd1^{-/-} mice. (FIG. 1C) Kaplan-Meier survival curve of Ctla4^{+/-} Pdcd1^{-/-} and littermate Ctla4^{+/+} Pdcd1^{-/-} mice stratified by mouse sex (n=24 and 29, male and female Ctla4^{+/-} Pdcd1^{-/-}; n=38 and 22, male and female Ctla4^{+/+} Pdcd1^{-/-}). Pairwise comparisons with Mantel-Cox Log-rank p-values less than 0.05 are denoted.

FIGS. 2A-D. Mono-allelic loss of Ctla4 in the absence of PD-1 leads to mortality with 50% penetrance. (FIG. 2A) Total body weight of symptomatic Ctla4+/− Pdcd1−/− and littermate control Ctla4+/+ Pdcd1−/− mice. (FIG. 2B) Total body weight of Ctla4+/− Pdcd1−/− and littermate control Ctla4+/+ Pdcd1−/− mice plotted as a function of age. Ctla4+/− Pdcd1−/− mice identified as requiring euthanasia are identified. Weights of Ctla4+/− Pdcd1−/− mice that were found dead without display of any symptoms were not able to be recorded and thus are not included here. (FIG. 2C) Serum chemistry of phenotypically unaffected aged 4-10 month old Ctla4+/− Pdcd1−/−, littermate control Ctla4+/+ Pdcd1−/− mice, and symptomatic Ctla4+/− Pdcd1−/− mice was performed. Significantly elevated levels of ALT, AST, and LDH were observed in symptomatic Ctla4+/− Pdcd1−/− mice. (FIG. 2D) A representative plot of total CTLA4 protein levels in in vitro stimulated T cells assessed by flow cytometry. CTLA4 expression levels in T cells derived from Ctla4+/+ Pdcd1−/− mice are plotted as a dotted line and that of Ctla4+/− Pdcd1−/− T cells plotted as a solid line. Quantitative data shown in FIG. 10D.

FIGS. 3A-E. Ctla4^{+/-} Pdcd1^{-/-} mice develop autoimmunity in multiple tissues. (FIG. 3A) Images of H&E stained FFPE pancreatic tissue sections from Ctla4^{+/-} Pdcd1^{-/-} and Ctla4^{+/+} Pdcd1^{-/-} mice. (FIG. 3B) Images of H&E stained FFPE heart tissue sections from Ctla4^{+/-} Pdcd1^{-/-} and Ctla4^{+/+} Pdcd1^{-/-} mice. (FIG. 3C, upper panel) Quantification of lymphoid infiltrate score in heart and pancreas tissue. (FIG. 3C, lower panel) Quantification of total T cells in pancreatic and heart tissue sections. (FIG. 3D) Graph shows quantification of total CD3 and lymphoid infiltrate scores in heart tissue. (FIG. 3E) Panels show representative histology images of heart tissue from Ctla4^{+/-} Pdcd1^{-/-} mice. H&E staining (top left) and CD3 immunohistochemistry (top right) images at low magnification are displayed. Arrows denote example areas of immune infiltration. Bottom rows: histology images at high magnification of immunohistochemistry staining of CD3 (T cell marker), CD4 (T cell marker), CD8 (T cell marker), and F4/80 (macrophage marker). This is an example of infiltration of each of these cell subtypes into the heart tissue of Ctla4^{+/-} Pdcd1^{-/-} mice.

FIG. 4. Molecular characterization of the immune response in Ctla4^{+/-} Pdcd1^{-/-} mice. T cell clonality assessed by TCR sequencing in lymph node, heart, and pancreatic tissue from Ctla4^{+/-} Pdcd1^{-/-} and Ctla4^{+/+} Pdcd1^{-/-} mice.

FIGS. 5A-F. Proteomic analyses reveal very subtle molecular changes due to single copy loss of Ctla4. (FIG. 5A) Principal component analysis plot of RPPA analysis of lymph nodes from Ctla4^{-/-}, Ctla4^{+/-}, and Ctla4^{+/+} mice. (FIG. 5B) The expression of significantly modulated proteins displayed as a heat map organized by two-way unsupervised hierarchical clustering. (FIG. 5C-E) Volcano plots of protein expression comparing wild-type versus heterozygous mice (FIG. 5C), wild-type versus Ctla4 knockout (FIG. 5D), and heterozygous versus Ctla4 knockout mice (FIG. 5E). (FIG. 5F) Expression values of specific proteins associated with cell cycle plotted as on a per mouse basis. Proteins with Tukey's multiple comparison p<0.05 between wild-type and heterozygous mice are denoted.

FIGS. 6A-E. Transcriptional analyses reveal little to no changes due to single copy loss of Ctla4 in context of functional PD-1. (FIG. 6A) Principal component analysis plot of Nanostring gene expression analysis of lymph nodes from Ctla4^{-/-} and littermate control mice (all homozygous wild-type Pdcd1). (FIG. 6B) The expression of significantly modulated proteins displayed as a heat map organized by two-way unsupervised hierarchical clustering. (FIGS. 6C-E) Volcano plots of protein expression comparing wild-type versus heterozygous mice (FIG. 6C), wild-type versus Ctla4 knockout (FIG. 6D), and heterozygous versus Ctla4 knockout mice (FIG. 6E).

FIG. 7. Ctla4^{+/-} Pdcd1^{-/-} mice are on a nearly pure C57BL6/J strain background and no segregating SNPs are associated with pathology. 100-SNP panel assessing strain background of pathogenic (affected) and non-pathogenic (unaffected) Ctla4^{+/-} Pdcd1^{-/-} mice. All tested mice harbored 97-100% C57BL6/J alleles.

FIG. 8. No segregating SNPs are associated with pathology of Ctla4+/− Pdcd1−/− mice. The frequency of non-homozygous B6 alleles across all mice are displayed for each SNP tested. non-homozygous B6 locus is defined as either heterozygous for B6/129 or homozygous for 129 alleles.

FIG. 9A-B. Generation and characterization transgenic mice with compound loss of function alleles of Ctla4 and Pdcd1. (FIG. 9A) Schematic of the breeding scheme to generate all potential combinations of Ctla4 and Pdcd1 loss of function mutant alleles. (FIG. 9B) Age of death of class I (mice that presented clinical symptoms prior to death) and class II (mice that died with prior presentation of symptoms) Ctla4+/− Pdcd1−/− mice.

FIG. 10A-D. Broad histological characterization of Ctla4+/− Pdcd1−/− mice reveals multi-tissue autoimmunity. (FIG. 10A) Scores of immune infiltration in the tissues denoted based on histological analyses of Ctla4+/− Pdcd1−/− and littermate control mice. (FIG. 10B) Number of foci of hepatic necrosis and/or inflammation per area of liver tissue analyzed by histology. (FIG. 10C) Score of lung adipose tissue atrophy in Ctla4+/− Pdcd1−/− and littermate control mice. (FIG. 10D) Quantitation of total CTLA-4 protein levels in in vitro stimulated T cells assessed by flow cytometry. CTLA-4 expression levels in T cells derived from Ctla4^{+/+} Pdcd1^{-/-} mice are plotted as a dotted line and that of Ctla4^{+/-} Pdcd1^{-/-} T cells plotted as a filled solid line.

FIG. 11A-B. Pathology of cardiac and pancreatic tissue in Ctla4^{+/-} Pdcd1^{-/-} mice. (FIG. 11A) Graphs show additional histology quantification from pancreas tissues. (FIG. 11B) Graph shows quantitation of serum antibody concentrations.

FIG. 12A-D. Mono-allelic loss of Ctla4 leads to decreased CTLA-4 protein. (FIG. 12A-D) Flow cytometry analysis total CTLA-4 in CD8 (A and B) and CD4 (C and D) T cells derived from Ctla4^{+/-} Pdcd1^{-/-}, littermate Ctla4^{+/+} Pdcd1^{-/-}, and C57BL6/J mice.

### DETAILED DESCRIPTION

T cell activation is tightly regulated via a wide range of mechanisms including negative co-stimulation, but the extent to which individual molecular mediators functionally interact remains unclear. CTLA4 and PD-1 are key negative regulators of T cell activation that utilize distinct molecular and cellular mechanisms. CTLA4 and PD-1 are both T cell negative costimulatory molecules whose function is to attenuate T cell activity. These molecules utilize distinct molecular mechanisms to carry out these functions. These molecular mechanisms are mediated by largely distinct cell signaling pathways (Wei et al., 2018). The primary function of CTLA4 is to compete for binding of B7 ligands (B7-1/CD80, B7-2/CD86), which leads to a reduction in CD28 positive costimulation and downstream PI3K/AKT signaling. The primary function of PD-1 is to inhibit proximal T cell receptor signaling upon binding to its ligands PD-L1/PD-L2; however, inhibition of CD28 signaling has also been reported as a significant function of PD-1 (Hui et al., 2017). Antibody-mediated blockade inhibits these functions by preventing ligand binding. This loss of signaling capacity and the consequent downstream biological events can be modeled by genetic means through the combination of loss of function alleles of Ctla4 and Pdcd1.

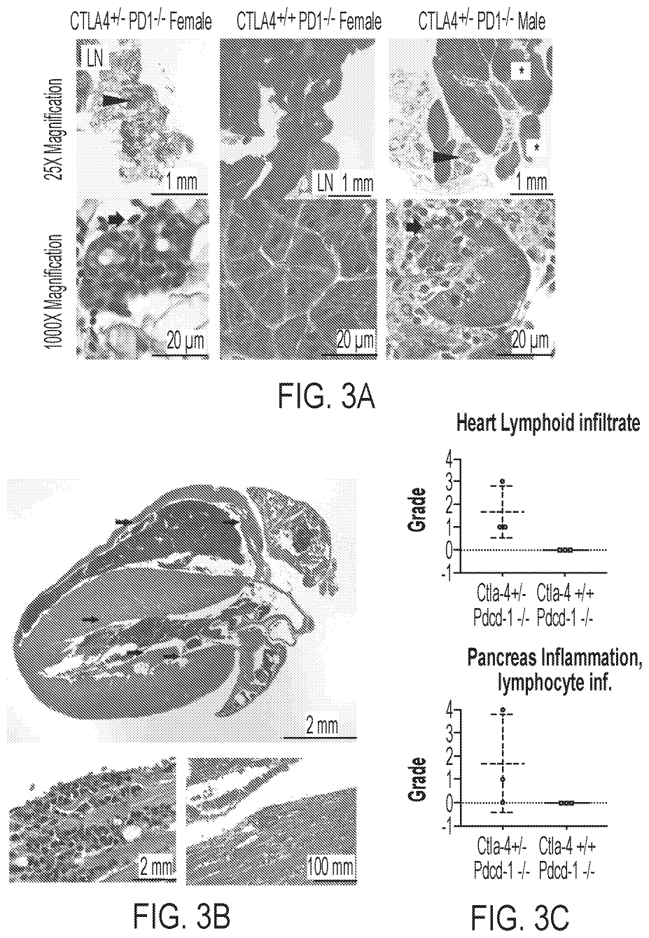

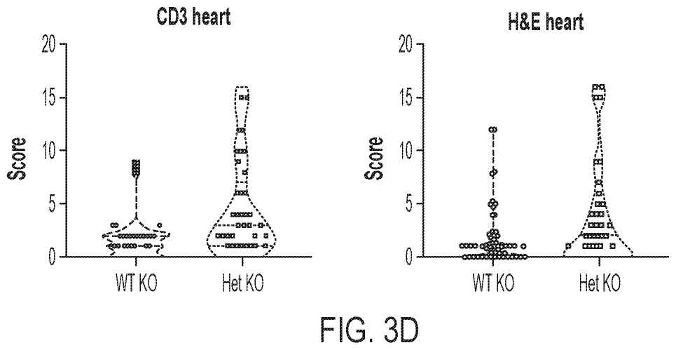

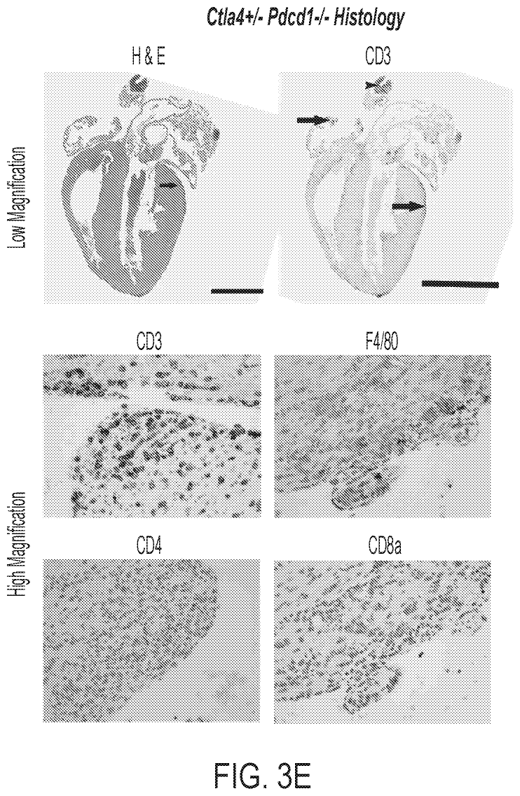

The inventors sought to understand whether regulatory mechanisms imposed by CTLA4 and PD-1 are functionally independent or dependent and found evidence of genetic interaction between Ctla4 and Pdcd1 (encoding PD-1) in mice. Mono-allelic loss of Ctla4 in the context of complete genetic absence of Pdcd1 led to death in approximately half of mice. Mortality was caused by autoimmunity in multiple tissues, including pancreas and heart (see, e.g., FIG. 3E). In contrast, no deaths or severe phenotypes were observed in littermate Ctla4^{+/+} Pdcd1^{-/-} or Ctla4^{+/-} Pdcd1^{+/-}. These data reveal that Ctla4 exhibits conditional haploinsufficiency in the context of a Pdcd1 null background. Together, these findings indicate that CTLA4 and PD-1 functionally interact and support a threshold model in which negative costimulatory molecules attenuate T cell activation in a dose-dependent and additive fashion.

This animal model will enable investigation into disease etiology and identification of factors that modulate the generation of immune related adverse events. This animal model develops autoimmunity in the heart and pancreas (as well as other organs), which is important because fatal myocarditis (inflammation of the heart) and type I diabetes (autoimmune destruction of the pancreas) are two types of rare but very serious complications associated with combination anti-CTLA4 and anti-PD-1 therapy in human patients. This model also appears to be able to model other types of autoimmune adverse events (e.g. gastrointestional) that are associated with checkpoint blockade. The transgenic mouse model described can be used as a model of combination anti-CTLA4 plus anti-PD-1 immune checkpoint blockade (i.e., treatment of monoclonal antibodies targeting T cell costimulatory receptors CTLA4 and PD-1). Genetic loss of PD-1 and single copy loss of CTLA4 models this therapy in an analogous scenario in which negative costimulatory activity is reduced and modeled. Thus, this genetic model that modulates CTLA4 and PD-1 recapitulates the phenomena of immune-related adverse events due to checkpoint blockade therapy. This is particularly notable because there are currently no animal models that faithfully recapitulate the adverse events associated with checkpoint blockade therapy. Combination anti-CTLA4 and anti-PD-1 checkpoint blockade therapy is currently approved for the treatment of melanoma and renal cell carcinoma (along with over 250 on-going clinical trials).

In addition, this mouse strain background is C57BL6/J, which is notable because of the widespread use of this background for tumor immunology studies and also because this background is normally very difficult to induce autoimmunity in, suggesting that this phenotype satisfies a high biological bar. Also, no exogenous antigens were introduced (e.g. transgenic, viral) and thus the antigens that are being recognized to drive this autoimmunity are self-antigens, as is presumed to be the case in the setting of patients that receive checkpoint blockade therapies. Furthermore, because multiple types of autoimmunity and immune-related disease etiologies arise in this animal model, this enables the investigation of the relationships between these diseases.

Thus, this mouse model can be used to understand how these adverse events are induced as well as to test the efficacy of therapies that aim to mitigate such adverse events and autoimmunity. Such investigation is likely necessary to design next-generation immunotherapies that retain therapeutic efficacy and reduce adverse events, particularly induction of rare, very serious autoimmunity.

### I. ASPECTS OF THE PRESENT INVENTION

A genetic interaction between the T cell negative costimulatory genes Ctla4 and Pdcd1 (encoding PD-1) is identified herein. This genetic interaction manifests as conditional haploinsufficiency of Ctla4 in the context of complete absence of Pdcd1, which leads to fatal systemic autoimmunity. From a fundamental perspective, this observation supports a threshold model of T cell activation in which multiple sources of T-cell receptor (TCR) signal perturbation can compound to result in aberrant T cell activation. This indicates that CTLA4 and PD-1 regulatory signals are functionally integrated and together provide a critical buffering system to restrain T cell activation. At the molecular level, decreases in the combined gene dosage of Pdcd1 and Ctla4 may limit the overall ability to attenuate T cell activation in a cell intrinsic manner.

In addition to the significant insights into basic mechanisms of T cell activation, these findings have notable clinical implications in the context of cancer immune checkpoint blockade. Combination anti-CTLA4 plus anti-PD-1 therapy is effective in multiple tumor types, including advanced melanoma and renal cell carcinoma. Anti-CTLA4 and anti-PD-1 immune checkpoint blockade are known to utilize distinct cellular mechanisms (Das et al., 2015; Wei et al., 2017). Taken in the context of the present findings, these distinct cellular mechanisms likely interact functionally, which may in part explain the enhanced efficacy of combination therapy versus monotherapies (Curran et al., 2010; Postow et al., 2015; Wolchok et al., 2013). Even more relevant to the present findings, immune checkpoint blockade therapy can induce serious immune-related adverse events as well as bona fide autoimmunity, such as myocarditis and type I diabetes, in rare instances. Ctla4^{+/-} Pdcd1^{-/-} mice provide a preclinical animal model with which to study autoimmunity induced by loss of CTLA4 and PD-1 signaling. This is particularly notable given that autoimmunity observed in this model closely reflect the myocarditis and type I diabetes that can arise following combination anti-CTLA4 plus anti-PD-1 checkpoint blockade therapy in human patients. Further mechanistic understanding provides the potential to distinguish and specifically modulate aspects of the immunological response that mediate efficacy and adverse events of immune checkpoint blockade therapy.

Interestingly, the autoimmunity that develops in Ctla4^{+/-} Pdcd1^{-/-} mice recurrently manifests in specific anatomical sites. Why particular tissue sites are more sensitive to autoimmunity induced by loss of PD-1 and CTLA4 signaling remains a critical open question. It is possible that tissue specific antigens from these sites render them particularly liable to autoimmune recognition in the absence of negative co-stimulation. Alternatively, it is possible that tissue sensitivity is due to functional differences between tissue-specific T_{reg} populations that have been previously observed (Legoux et al., 2015).

Notably, heterozygous germ line loss of function alleles of CTLA4 lead to immune dysregulation with highly variable clinical presentation (Kuehn et al., 2014; Schubert et al., 2014). This indicates that single copy loss of CTLA4 in humans is pathogenic and furthermore, is strongly suggestive of genetic interaction with other genetic and/or environmental factors. It is also possible that single copy loss of CTLA4 in humans or in mice (in the absence of PD-1) lowers the T cell activation threshold to the level at which tonic TCR signaling can reach, and thus stochastic processes may explain the variance in clinical presentation in CTLA4 deficient humans and mice. An outstanding question is the extent to which other T cell costimulatory molecules or molecules involved in their function genetically interact with Pdcd1 and Ctla4. For example, patients harboring loss of function alleles of LRBA, an important regulator of CTLA4 trafficking, present with similar autoimmune phenotypes as patients harboring loss of function CTLA4 (Besnard et al., 2018; Hou et al., 2017).

In addition to the key finding of genetic interaction between CTLA4 and PD-1, the present findings also suggest that simultaneous genetic deficiency of Ctla4 and Pdcd1 is embryonic lethal. This is surprising given that αβ T cells do not emerge until after birth (Havran and Allison, 1988). The mechanism through which this occurs remains unclear, however there are two main possibilities, both of which are quite intriguing. The first possibility is that CTLA4 and PD-1 restrict activation of yδ T cells, which emerge as early as E14 during embryonic development. The second possibility is that CTLA4 and PD-1 may have as yet unidentified non-immunological functions during development.

In conclusion, the present findings reveal genetic interaction between Ctla4 and Pdcd1. This provides definitive evidence for functional interaction between the regulatory mechanisms of CTLA4 and PD-1. Furthermore, this provides a robust animal to model immune-related adverse events induced by combination anti-CTLA4 plus anti-PD-1 immune checkpoint blockade therapy.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

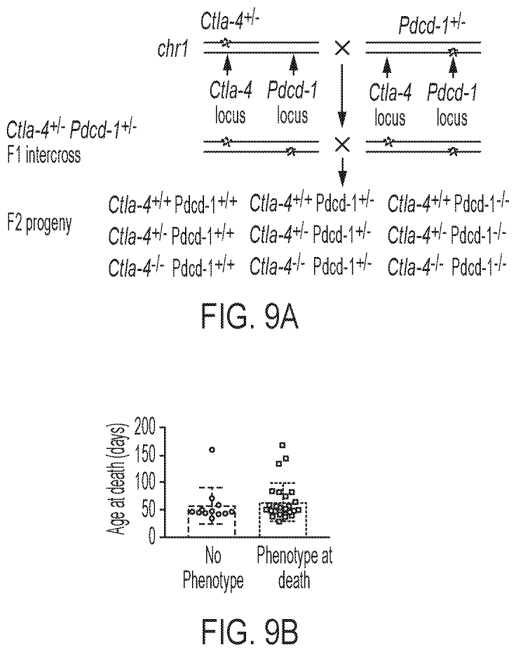

Mice. Ctla4^{tm1All} mice (Chambers et al., 1997) were bred to Pdcd1 knockout mice (Pdcd1^{tm1.1Shr}) (Keir et al., 2007), which were purchased from The Jackson Laboratory (021157). Pdcd1 knockout mice were backcrossed once to C57BL/6J prior to this cross. Resulting F1 Ctla4^{+/-} Pdcd1^{+/-} mice were intercrossed to produce all possible combinations of wild-type and mutant alleles of the two genes. This first breeding scheme specifically utilized F1 mice derived from the cross of Ctla4^{+/-} (which are wild-type for Pdcd1) and Pdcd1^{-/-} mice (which are wild-type for Ctla4). This breeding scheme ensures that mutant alleles of Ctla4 and Pdcd1 in F1 mice are in trans, and thus the recombination frequency can be calculated (FIG. 9A). The genetic distance between Ctla4 and Pdcd1 was calculated by assessing the number of recombination and total events in this cross. The observed recombination frequency of 17.03 cM closely aligned with the 16.89 centi-Morgan genetic distance between Ctla4 and Pdcd1 reported by the Mouse Phenome Database (MPD, RRID:SCR_003212) (Bogue et al., 2018). This estimated genetic distance was consistent with the observed recombination frequency in this breeding scheme.

To verify findings from the first breeding scheme, a second related breeding scheme was utilized. Importantly, this breeding approach utilized different genotypes, the mutant alleles could either be in cis or trans, and the approach would generate Ctla4^{+/-} Pdcd1^{-/-} (experimental) and Ctla4^{+/+} Pdcd1^{-/-} (control littermates) in a 1:1 ratio. This allows for the generation of many more Ctla4^{+/-} Pdcd1^{-/-} mice than in the initial breeding approach. Specifically, male Ctla4^{+/-} Pdcd1^{-/-} and female Ctla4^{+/+} Pdcd1^{-/-} were bred. Female Ctla4^{+/+} Pdcd1^{-/-} were used to eliminate the possibility that the autoimmunity observed in Ctla4^{+/-} Pdcd1^{-/-} might affect fetal-maternal tolerance or the ability to produce viable litters.

For the generation of survival curves, events were defined as either death (i.e. mice found dead) or identification of mice by veterinary staff as requiring euthanasia (e.g. due to lethargy, moribund, dyspnea). For mice identified as requiring euthanasia, the date of death was defined as the day the mouse was flagged by veterinary staff. Animal phenotypes associated with mortality were identified and reported by veterinary staff. Mice utilized for breeding were censored from survival analyses at the time that they were utilized for this purpose.

All mice were housed at The University of Texas MD Anderson Cancer Center South Campus Vivarium, an AAALAC-accredited specific pathogen-free animal facility. All experiments were performed in accordance with The University of Texas MD Anderson Cancer Center Institutional Animal Care and Use Committee (IACUC) guidelines.

Genotyping. Genomic DNA was isolated using Direct-to-PCR digest mix and polymerase chain reaction (PCR) based genotyping was performed for Ctla4 and Pdcd1 knockout mice. Primers are provided in Table 1. Ctla4^{tm1All} mice were genotyped as previously described (Chambers et al., 1997). The expected band sizes for the Ctla4 wild-type and mutant alleles are ˜75 and ˜150 bp, respectively. Pdcd1 knockout mice were genotyped as previously described (Keir et al., 2007). The expected band sizes for the Pdcd1 wild-type and mutant alleles are 418 and 350 bp, respectively.

TABLE 1
Primers for genotyping.

| | | | SEQ |
|---|---|---|---|
| | | | ID |
| | | Sequence | NO: |
| CTLA4 | | 5′ AAACAACCCCAAGCTAACTGCGACAAGG 3′ | 1 |
| primers | | 5′ CCAGAACCATGCCCGGATTCTGACTTC 3′ | 2 |
| | | 5′ CCAAGTGCCCAGCGGGGCTGCTAAA 3′ | 3 |
| Pdcd1 | PD1 KO | 5′ CACTATCCCACTGACCCTTCA 3′ | 4 |
| primers | common | | |
| | PD1 KO | 5′ AGAAGGTGAGGGACCTCCAG 3′ | 5 |
| | WT | | |
| | PD1 KO | 5′ CACAGGGTAGGCATGTAGCA 3′ | 6 |
| | Mut rev | | |

SNP typing. Crude genomic DNA lysate was submitted to The University of Texas MD Anderson Cancer Center Laboratory Animal Genetics Services core facility for SNP-typing using a 100-marker panel. For the purpose of determining whether genetic variants associate with autoimmunity, ‘unaffected’ mice were defined as mice that did not manifest any symptoms or die within 6 months of age and ‘affected’ mice were defined as class I mice that manifested symptoms and succumbed to disease.

Pathology analyses. Animal necropsies were performed by personnel in The University of Texas MD Anderson Cancer Center veterinary medical histology laboratory or in the Allison laboratory. Automated serum chemistry analysis using a Cobas Integra 400Plus (Roche Diagnostics, Risch-Rotkreuz, Switzerland) was performed on a blood sample collected at euthanasia. Formalin-fixed tissues were processed routinely into paraffin blocks, sectioned at 5 microns, and stained with hematoxylin and eosin. Additional sections were used for immunohistochemical (IHC) staining of particular tissues of interest, using an antibody directed against CD3 (ab16669, Abcam, Cambridge, Mass.), followed by secondary reagents for chromogenic detection (Bond Polymer Refine Detection system, DS9800, Leica, Buffalo Grove, Ill.). Stained sections were examined by a veterinary pathologist using a Leica DM2500 microscope with Leica DFC495 camera and Leica Application Suite v4.12 software. Histologic changes were scored using a semi-quantitative scale, with 0=no lesion to 4=severe lesion.

Flow cytometry. Single cell suspensions from lymph nodes were prepared by mashing pooled inguinal, axillary, and brachial lymph nodes through a 70 um filter using the back of a plastic syringe into RPMI-1640 supplemented with 10% FBS and 1% Penicillin Streptomycin. A 96-well flat bottom plate was coated 200 ul per well of 1 ug/ml anti-CD3E and 2 ug/ml anti-CD28 in PBS overnight at 4° C. the previous night. Cells were then stained with CellTrace Violet Proliferation kit per the manufactures protocol (Invitrogen, C34557). Triplicates of each sample were plated 10⁶ cells/mL per well in 200 ul of RPMI-1640 supplemented with 10% FBS, sodium pyruvate, 0.1% b-ME, and P/S and incubated at 37° C. for 46 hours. Cells were then transferred to a U-bottom 96-well plate and washed twice with FACS buffer and incubated with 2% of each bovine, murine, rat, hamster, and rabbit serum PBS with 25 mg/mL 2.4G2 antibody at 4° C. for 10 min prior to surface staining with an antibody cocktail at 4° C. for 30 min in a 50 mL volume. Cells were washed twice with FACS buffer then fixed and permeabilized using the FoxP3 fix and permeabilization kit according to manufacturer's protocol (eBioscience). Cells were subsequently stained with an intracellular antibody cocktail at room temperature for 30 min. Cells were then washed twice with Foxp3 permeabilization buffer, then twice with FACS buffer, and analyzed on a LSRII (BD).

For surface stain (restim) the following antibodies were used: LIVE/DEAD™ Fixable Blue Dead Cell Stain L23105 (ThermoFisher); BV786 Hamster Anti-Mouse CD3e (clone 145-2C11, 564379 (BD)); Brilliant Violet 605 anti-mouse TCR 13 chain Antibody (clone)H57-597, 109241(BioLegend)); Brilliant Violet 650 anti-mouse CD19 Antibody (clone 6D5, 115541 (BioLegend)); FITC Anti-mouse CD4 Antibody (clone RM4.5, 11-0042-82 (ebio)); PE anti-mouse CD152 Antibody (clone UC10-4B9, 106306 (BioLegend)); PE Armenian Hamster IgG Isotype Ctrl Antibody (clone HTK888, 400908 (Biolegend); APC Anti-mouse CD8a Antibody (clone 53-6.7, 17-0081-82 (ebio)); and Alexa Fluor 700 Anti-mouse CD45.2 Antibody, (clone 104, 56-0454-82 (ebio)). For IC stain (restim) the following antibodies were used: BV786 Hamster Anti-Mouse CD3e (clone 145-2C11, 564379 (BD)); Brilliant Violet 605 anti-mouse TCR (3 chain Antibody (clone H57-597, 109241(BioLegend)); FITC Anti-mouse CD4 Antibody (clone RM4.5, 11-0042-82 (ebio)); PE anti-mouse CD152 (CTLA-4) Antibody (clone UC10-4B9, 106306 (BioLegend)); PE Armenian Hamster IgG Isotype CTLA-4 Ctrl Antibody (clone HTK888, 400908 (BioLegend)); and APC Anti-mouse CD8a Antibody (clone 53-6.7, 17-0081-82 (ebio)).

Luminex cytokine and chemokine assessment. Serum was collected from Ctla4^{+/-} Pdcd1^{-/-} and control littermate mice (including both Ctla4^{+/+} Pdcd1^{-/-} mice and mice competent for both CTLA4 and PD-1 such as Ctla4^{+/-} Pdcd1^{-/-} mice) from both breeding schemes described above. Briefly, blood was collected by terminal cardiac puncture, allowed to coagulate at room temperature, centrifuged at 8,000 g for 10 minutes, supernatant serum collected and snap frozen in liquid nitrogen prior to storage at −80 degrees Celsius. Serum levels of antibodies cytokine and chemokine were assessed using the Cytokine & Chemokine 36-plex Mouse ProcartPlex luminex assay (ThermoFisher Scientific) per manufacturer's protocol. All samples were analyzed in parallel in a single batch for each respective analysis. Serum samples were diluted 1:10,000 for analysis of serum antibody levels.

Reverse phase proteomic array analysis. Lymph nodes from 16-day-old Ctla4 knockout and littermate control mice were snap frozen for subsequent analysis. Tissue samples were lysed in 1% Triton X-100, 50 mM HEPES, pH 7.4, 150 mM NaCl, 1.5 mM MgCl₂, 1 mM EGTA, 100 mM NaF, 10 mM Na pyrophosphate, 1 mM Na₃VO₄, 10% glycerol, with freshly added protease inhibitors (Roche, 05056489001) and phosphatase inhibitors (Roche, 04906837001) and homogenized using a Precellys homogenizer (Bertin Instruments). Samples were diluted in sample buffer (10% glycerol, 2% SDS, 62.5 mM Tris-HCl, BME, pH 6.8) prior to array printing and analysis. Signal was quantified using Array-Pro Analyzer software (MediaCybernetics) and normalized using a “Supercurve fitting” approach developed at MD Anderson Cancer Center for the RPPA Core Facility. Normalized linear values were analyzed in Excel (Microsoft) using two-tailed T-test assuming unequal variance and plotted using Prism 6.0 (GraphPad).

Nanostring mRNA analysis. Lymph nodes were dissected from Ctla4 knockout and littermate control mice. RNA was extracted from lymph nodes using (Qiagen). 100 ng RNA was analyzed using the Mouse Immunology Code set panel on the nCounter platform (Nanostring). Values were normalized on a per sample basis using housekeeping genes within the panel. Heat maps of Nanostring gene expression and RPPA proteomic data were generated in R utilizing a Pearson distance matrix and Ward's minimum variance method. Only differentially expressed genes, defined by a false discovery rate of 5% with a one-way ANOVA comparison between genotypes, were plotted.

Statistics. Statistical analyses were performed in Prism 7.0 or 8.0 (GraphPad Software, San Diego, Calif.), unless otherwise noted. Normalized linear values of RPPA data were analyzed in Excel (Microsoft) using two-tailed T-test assuming unequal variance and plotted.

### Example 1—Lethal Haploinsufficiency of Ctla4 in the Genetic Absence of Pdcd1

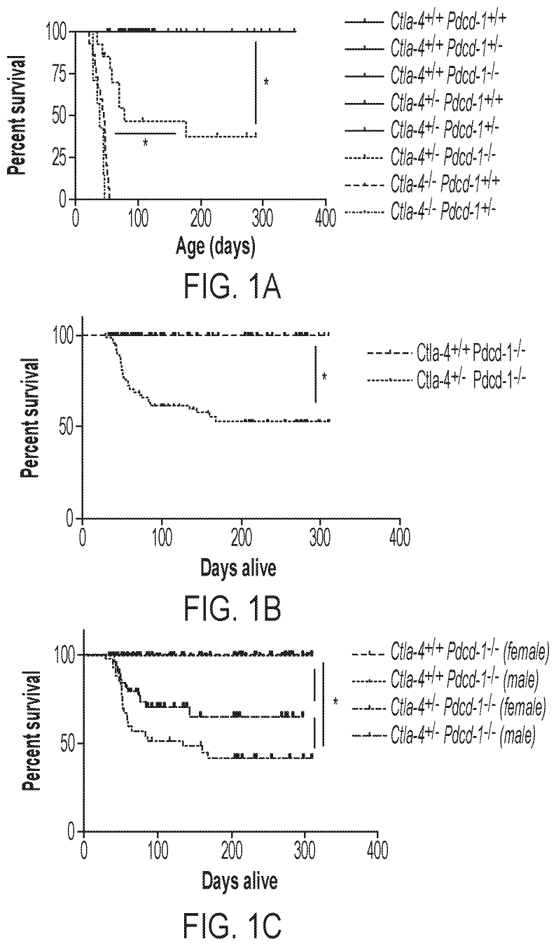

To test whether there is a genetic interaction between Ctla4 and Pdcd1, Ctla4 and Pdcd1 (encoding PD-1) knockout transgenic mice were crossed. Murine Ctla4 and Pdcd1 are genetically linked with a genetic distance of 16.89 cM based on estimations from the Mouse Phenome Database (Bogue et al., 2018). A heterozygous intercross-breeding scheme (see Materials and Methods) was used that allowed for the generation of all possible permutations of mutant alleles from a single cross and for estimation of the observed recombination frequency between Ctla4 and Pdcd1 at 17.03 cM. Surprisingly, approximately 50% of Ctla4^{+/-} Pdcd1^{-/-} spontaneously died within 3 months of age (FIG. 1A). In contrast, related control littermates (e.g. Ctla4^{+/+} Pdcd1^{-/-} and Ctla4^{+/-} Pdcd1^{+/-}) exhibited no overt phenotypes and no deaths were observed in these groups. The lack of an overt phenotype in Ctla4^{+/+} Pdcd1^{-/-} mice is consistent with prior observations (Nishimura et al., 1999). In addition, death of all of the Ctla4^{-/-} Pdcd1^{+/+} mice is consistent with the initial characterization of CTLA4 deficient mouse strains (Chambers et al., 1997; Tivol et al., 1995; Waterhouse et al., 1995). Interestingly though, monoallelic loss of Pdcd1 accelerated the fatal lymphoproliferation induced by loss of CTLA4 (FIG. 1A). This suggests that Pdcd1 gene dosage modifies the phenotype and lymphoproliferative disease of CTLA4 deficient mice. These observations contrast the absence of any PD-1 haploinsufficiency in Ctla4^{+/+} Pdcd1^{+/-} or Ctla4^{+/-} Pdcd1^{+/-} mice.

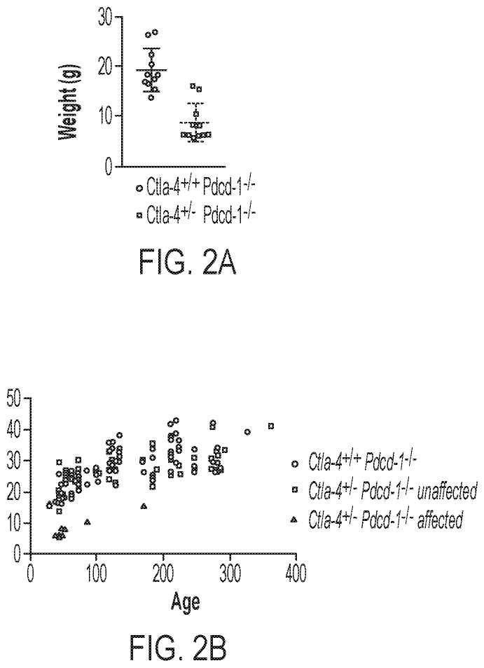

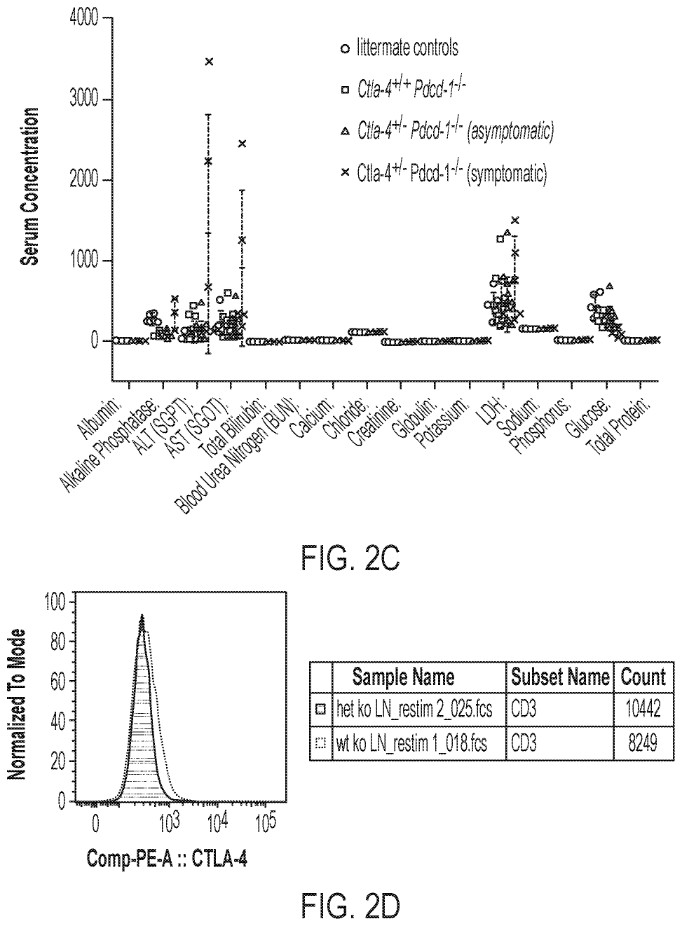

Of particular interest was the surprising spontaneous death of Ctla4^{+/-} Pdcd1^{-/-} mice as this provides strong evidence of genetic interaction. To further confirm this observation, Ctla4^{+/-} Pdcd1^{-/-} and Ctla4^{+/+} Pdcd1^{-/-} littermate mice were generated using a different breeding scheme (see Materials and Methods). This second approach yielded remarkably similar findings with approximately 50% of Ctla4^{+/-} Pdcd1^{-/-} mice spontaneously dying while no deaths were observed in littermate Ctla4^{+/+} Pdcd1^{-/-} mice (FIG. 1B). Mice spontaneously died or became moribund between 2-6 months of age, with a variety of observed clinical symptoms (Table 2, see Materials and Methods). The onset of these specific phenotypes is not necessary per say for the fatal autoimmunity observed however, given that of mice with and without prior presentation of these symptoms has similar latency of death (FIG. 9B). Interestingly however, male and female Ctla4^{+/-} Pdcd1^{-/-} mice died at significantly different frequencies (FIG. 1C). This indicates that the observed conditional haploinsufficiency of Ctla4 is sex-dependent, with female mice dying at higher frequency than male mice. This sex imbalance is consistent with the increased overall risk of irAEs in female patients receiving anti-CTLA-4 ICI (Valpione et al., 2018). Mortality was preceded by overt, non-specific clinical signs (e.g. reduced weight gain, ataxia, dyspnea) beginning as early as 1 month of age in approximately two-thirds of mice that died. Reflective of the severity of this phenotype the total body weight of Ctla4^{+/-} Pdcd1^{-/-} mice displaying clinical signs was significantly lower than that of Ctla4^{+/-} Pdcd1^{-/-} mice not displaying clinical signs (FIG. 2B).

Together these observations indicate that Pdcd1 and Ctla4 exhibit strong genetic interaction. Specifically, there is a dramatic Ctla4 conditional haploinsufficiency, which manifests only in the context of genetic deletion of Pdcd1 and leads to spontaneous deaths (FIGS. 1A-B).

TABLE 2
Phenotypes associated with mortality of Ctla4^{+/-} Pdcd1^{-/-} mice. The
absolute number and relative frequency of phenotypes associated with Ctla4^{+/-}
Pdcd1^{-/-} mice that were found dead or identified as requiring euthanasia.

| | No | | | | | | | | | |
|---|---|---|---|---|---|---|---|---|---|---|
| | observed | | Thin/ | | Rough | | | | | Abdominal |
| | phenotype | Small | Emaciated | Hunched | coat | Lethargic | Paresis | Hyperpnea | Ataxia | swelling |
| Frequency | 45.45 | 9.09 | 50 | 45.45 | 13.63 | 31.81 | 4.54 | 13.63 | 9.09 | 4.54 |

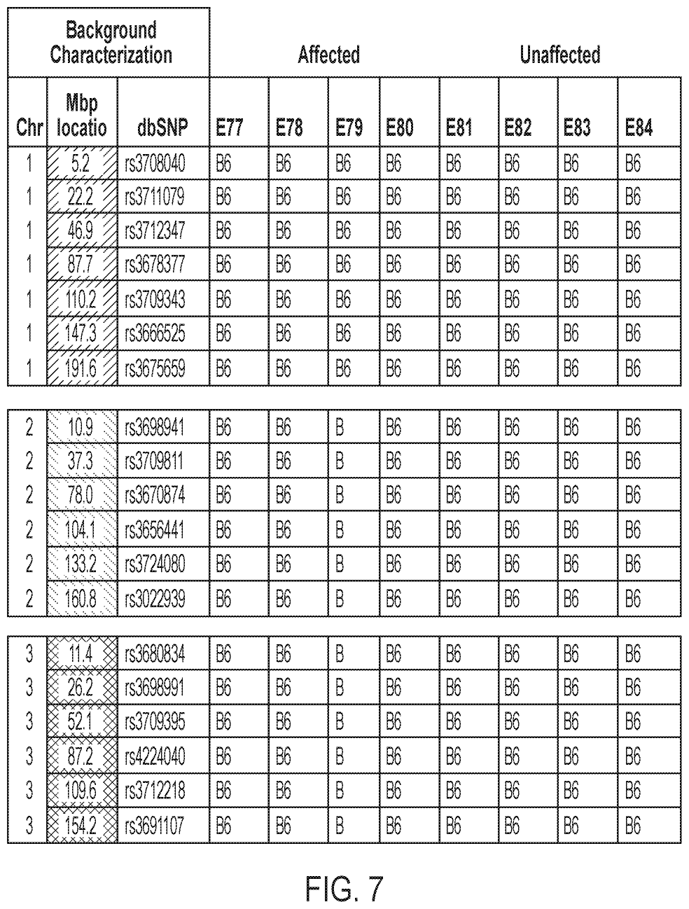

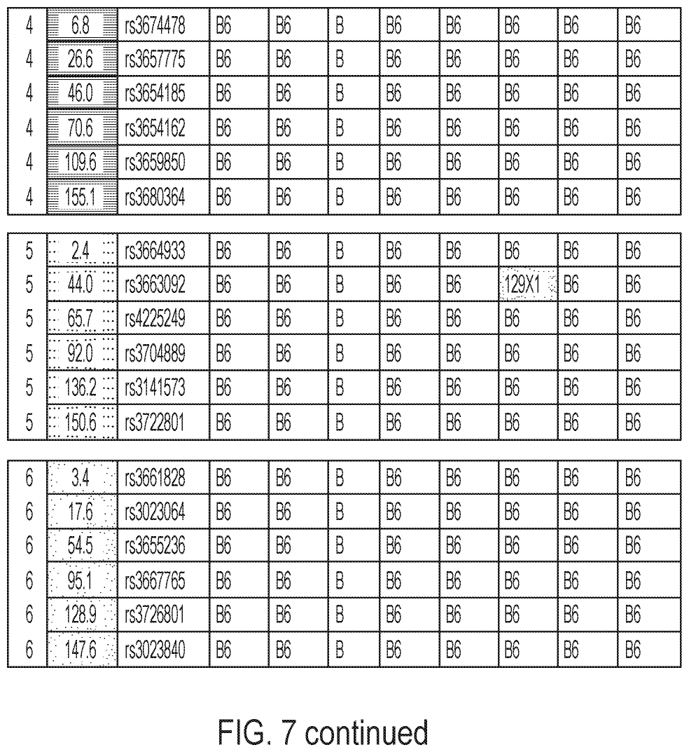

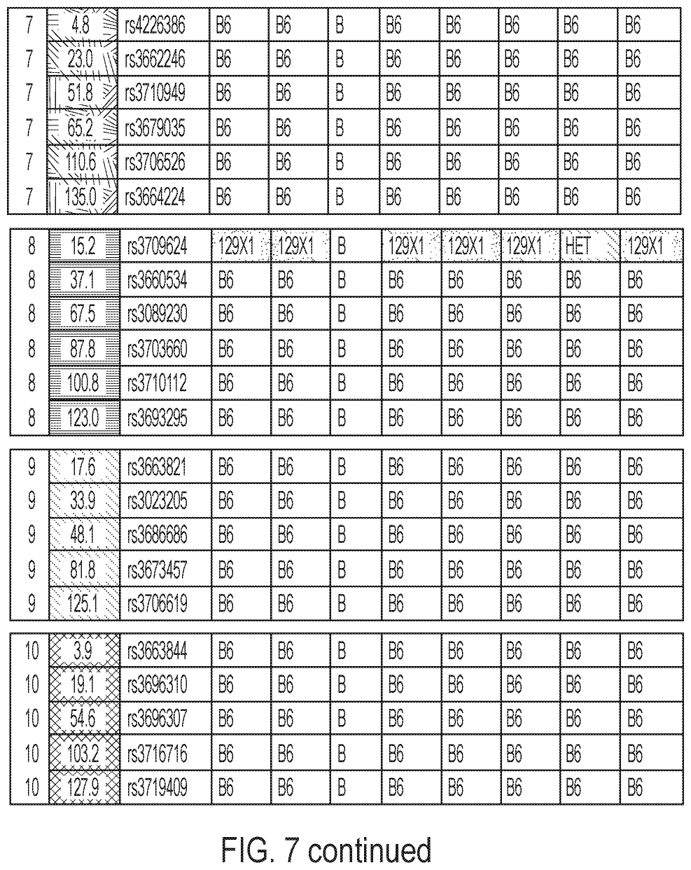

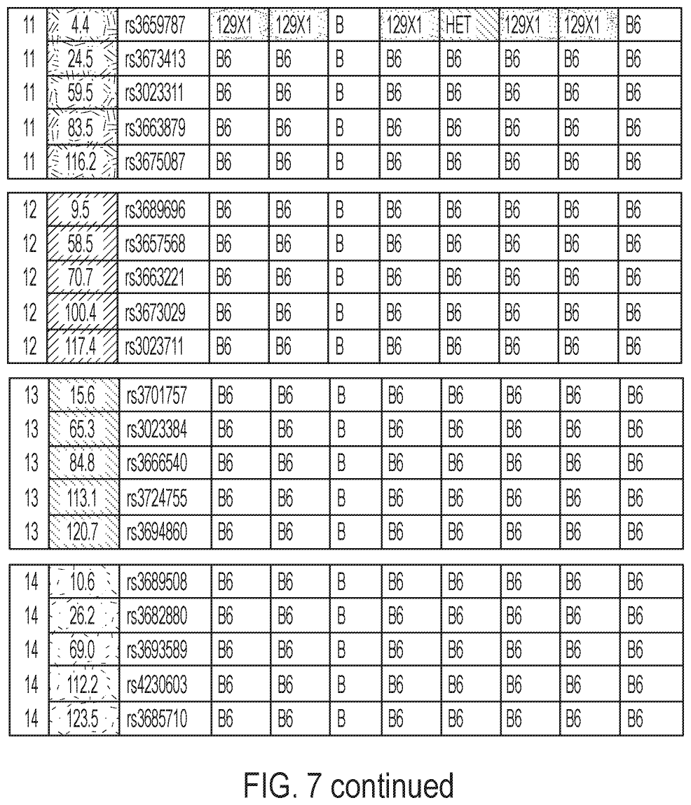

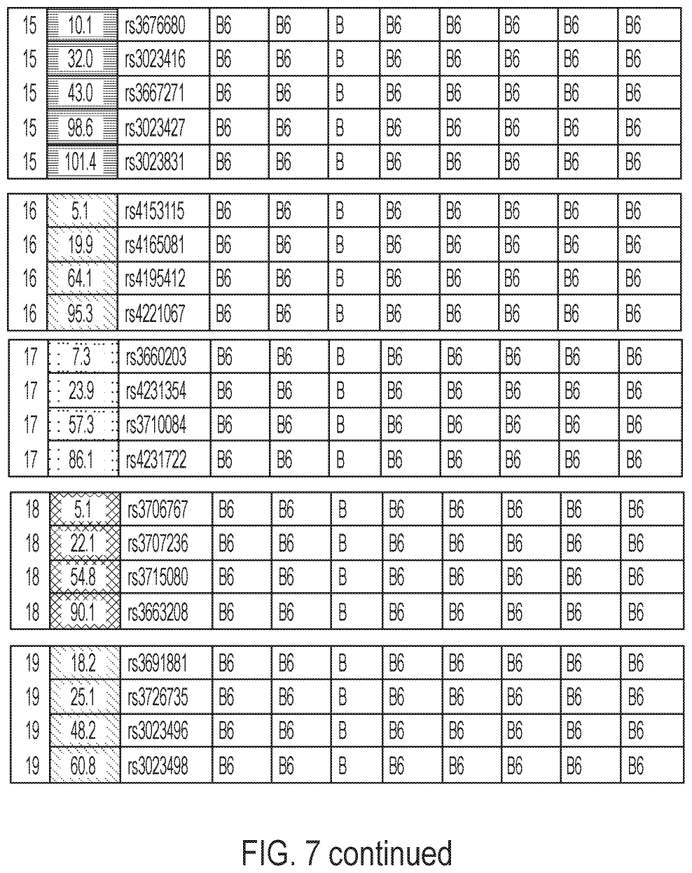

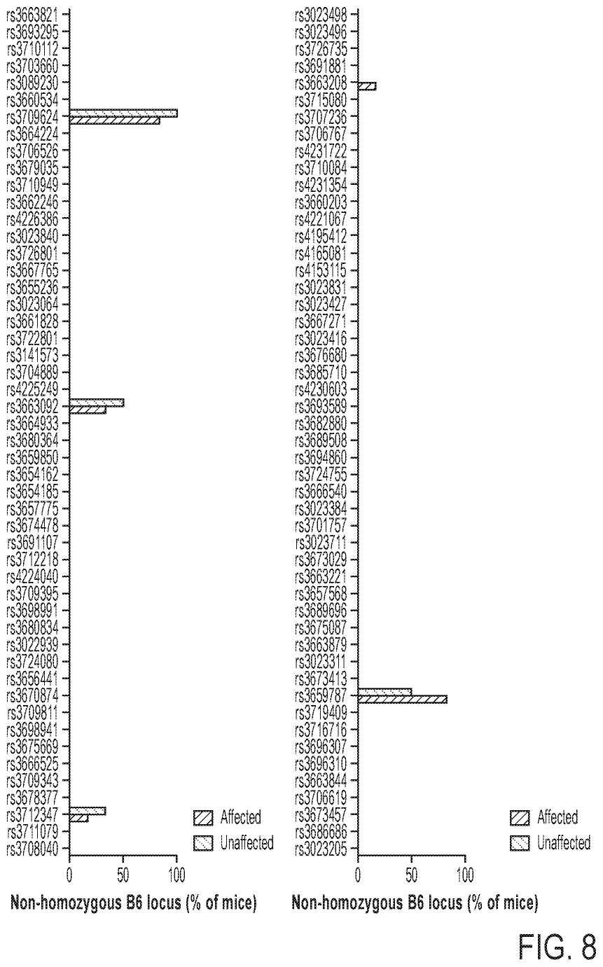

Given that only 50% of Ctla4^{+/-} Pdcd1^{-/-} mice die, it is likely that additional genetic or environmental factors modulate the penetrance of Ctla4 conditional haploinsufficiency. Whether subtle genetic differences could underlie this dichotomy was investigated. All tested mice were 97-100% C57BL6/J based on a 100-marker single nucleotide polymorphism typing panel, with no associations between any segregating alleles with phenotypic manifestation were observed (FIG. 7 and FIG. 8).

It was then determined whether Ctla4^{+/-} Pdcd1^{-/-} mice that did not spontaneously die and reached the age of the survival plateau (approximately 6 months) harbored subtle autoimmunity, which is biologically relevant but not sufficient to cause death. In contrast, the total body weights of affected Ctla4^{+/-} Pdcd1^{-/-} mice were significantly reduced compared to either phenotypically normal Ctla4^{+/-} Pdcd1^{-/-} or Ctla4^{+/+} Pdcd1^{-/-} mice (FIG. 2A). In contrast, the total body weight of phenotypically normal Ctla4^{+/-} Pdcd1^{-/-} and control littermate Ctla4^{+/+} Pdcd1^{-/-} mice were not significantly different (FIG. 2B). To further investigate whether biological, basic serum chemistry was performed on samples from aged ([4+] months) unaffected Ctla4^{+/-} Pdcd1^{-/-} and Ctla4^{+/+} Pdcd1^{-/-} mice. Interestingly, no significant differences between genotypes were observed (FIG. 2B). This suggests that unaffected mice (defined as the absence of phenotypic decline leading to death) do not develop significantly increased autoimmunity, at least detectable by markers of systemic tissue damage (e.g., LDH) or visual inspection. Notably, however, analyses of serum chemistries phenotypically affected Ctla4+/− Pdcd1−/− mice revealed evidence of tissue damage with significantly elevated serum levels of ALT, AST, and LDH as well as decreased glucose levels (FIG. 2C). The findings suggest that tissue destruction in Ctla4+/− Pdcd1−/− mice, and is detectable in peripheral blood.

These data support a model in which environmental factors modulate the penetrance and development of fatal phenotypes in Ctla4^{+/-} Pdcd1^{-/-} mice. From a fundamental perspective, this observation supports a threshold model of T cell activation in which multiple sources of TCR signal perturbation are integrated to regulate T cell activation. In the case of Ctla4^{+/-} Pdcd1^{-/-} mice, the threshold for activation is significantly lowered such that additional subtle inputs, which are normally buffered, are sufficient to induce aberrant T cell activation and autoimmunity. A prediction of this model is that T cells derived from Ctla4^{+/-} Pdcd1^{-/-} mice have decreased levels of CTLA-4 compared to T cells derived from Ctla4^{+/+} Pdcd1^{-/-} mice. To confirm that single copy loss of Ctla4 leads to a decrease in available CTLA-4 protein, we assessed total CTLA-4 protein expression in activated T cells from Ctla4^{+/-} Pdcd1^{-/-} and littermate control Ctla4^{+/+} Pdcd1^{-/-} mice. Notably, flow cytometry analysis of in vitro stimulated T cells are suggestive of lower levels of CTLA-4 protein in T cells derived from Ctla4^{+/-} Pdcd1^{-/-} mice compared to T cells derived from Ctla4^{+/+} Pdcd1^{-/-} mice (FIG. 2D).

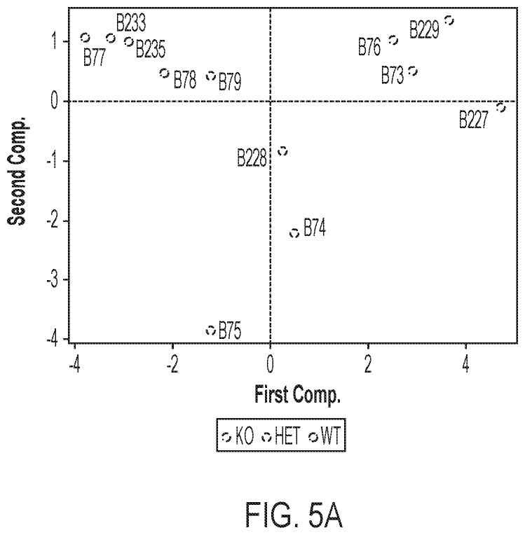

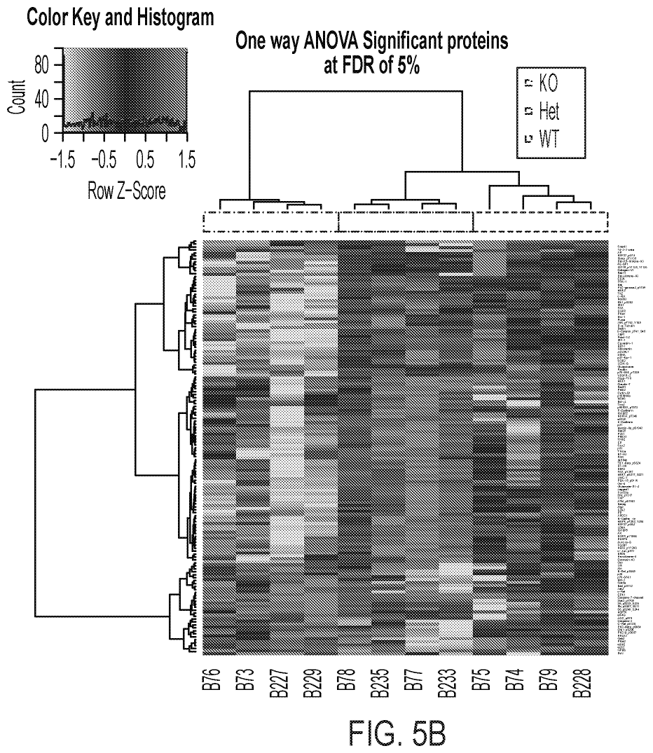

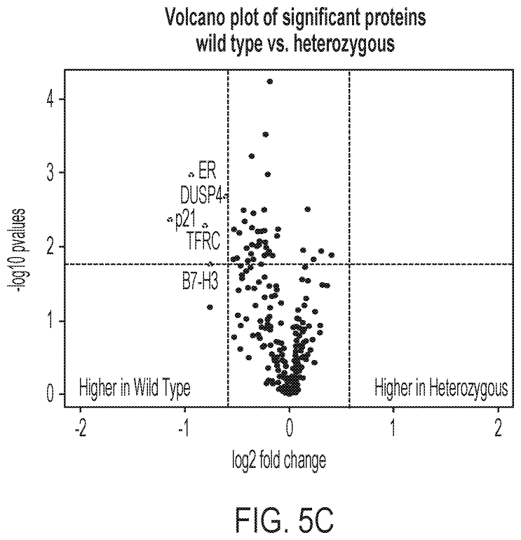

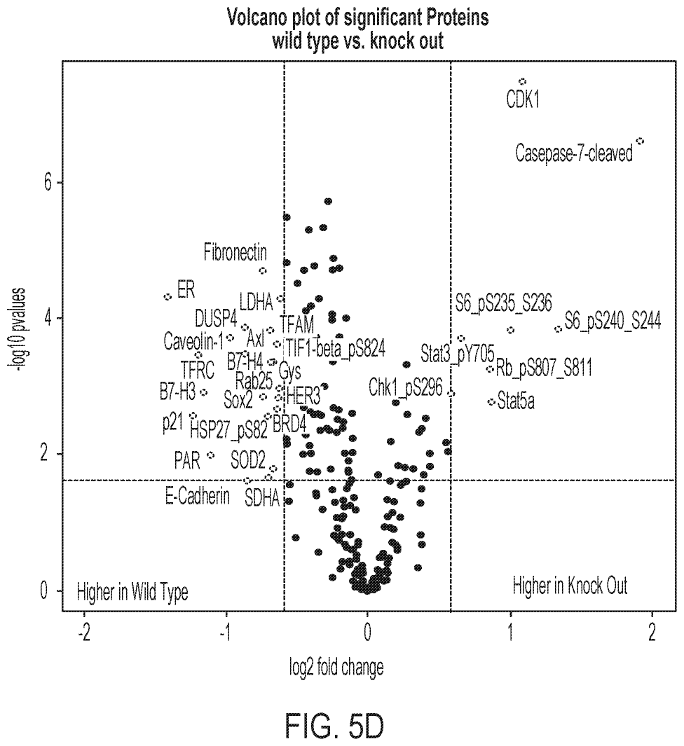

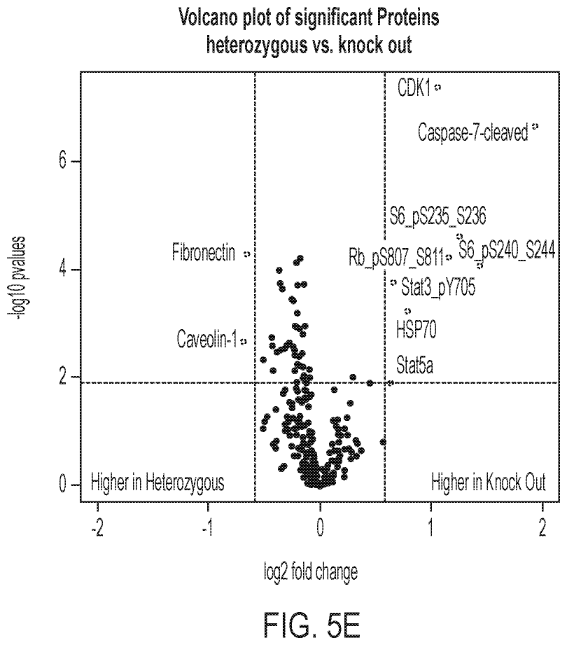

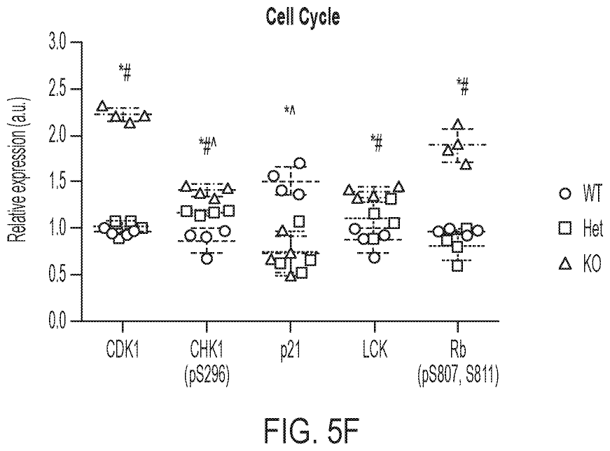

Taken in the context of prior findings that Ctla4^{+/-} mice do not display any haploinsufficiency at either the organismal or cellular level, these findings indicate that PD-1 negative co-stimulation is sufficient to functionally buffer mono-allelic loss of Ctla4. Consistent with this notion, few differences were observed in transcriptional and proteomic analyses of lymph nodes from Ctla4^{+/+} and Ctla4^{+/-} mice where dramatic changes were observed in Ctla4^{-/-} mice (FIGS. 5 & 6).

Interestingly, these results contrast the findings from mass cytometry profiling of similar tissues from Ctla4^{+/+} and Ctla4^{+/-} mice (with no perturbation in Pdcd1), in which no differences in cellular phenotype or frequency were detected. This suggests that at least in homogeneous inbred murine strains, additional regulatory molecular mechanisms can buffer against perturbations in signaling caused by single copy loss of Ctla4. However, in the context of additional perturbations, such as genetic loss of PD-1, functional defects in T cell regulation due to mono-allelic Ctla4 can manifest due to a loss of buffering capacity.

### Example 2—Ctla4^{+/-} Pdcd1^{-/-} mice develop multi-tissue autoimmunity

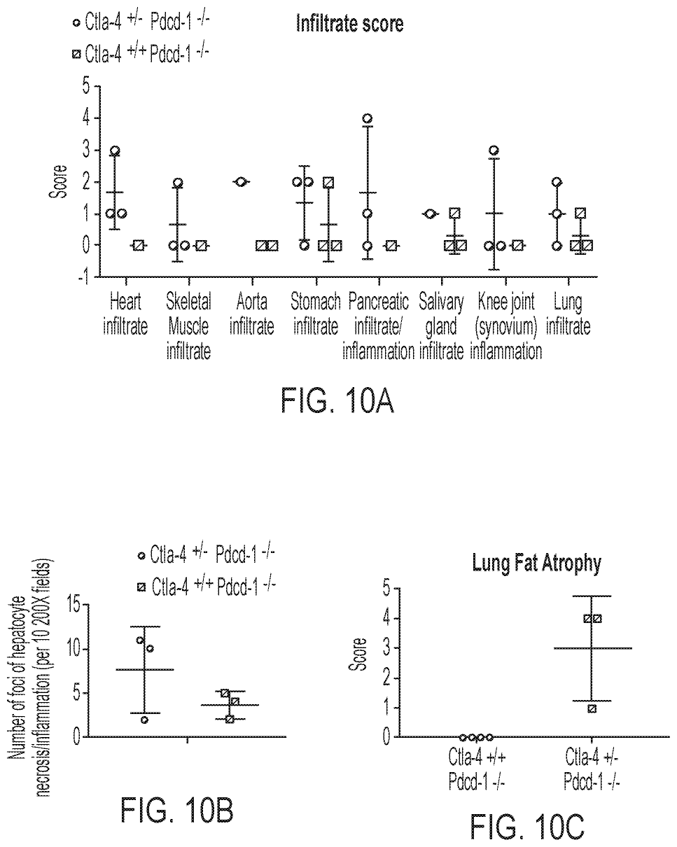

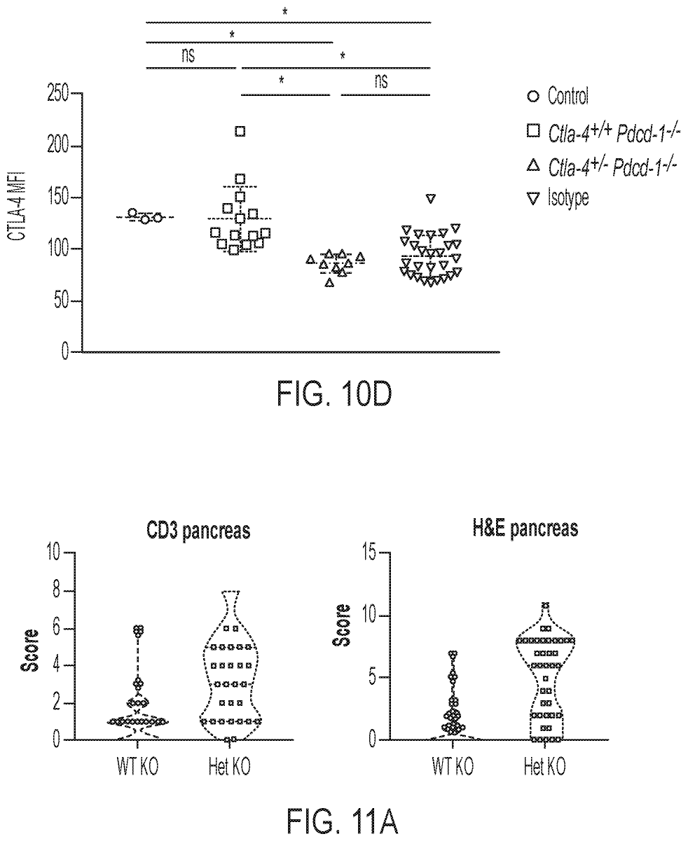

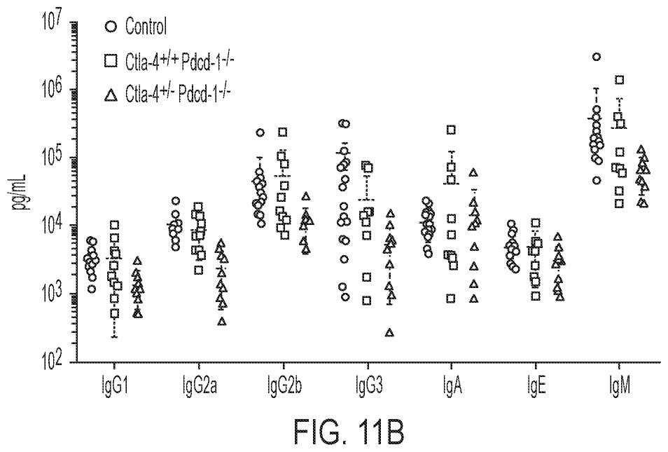

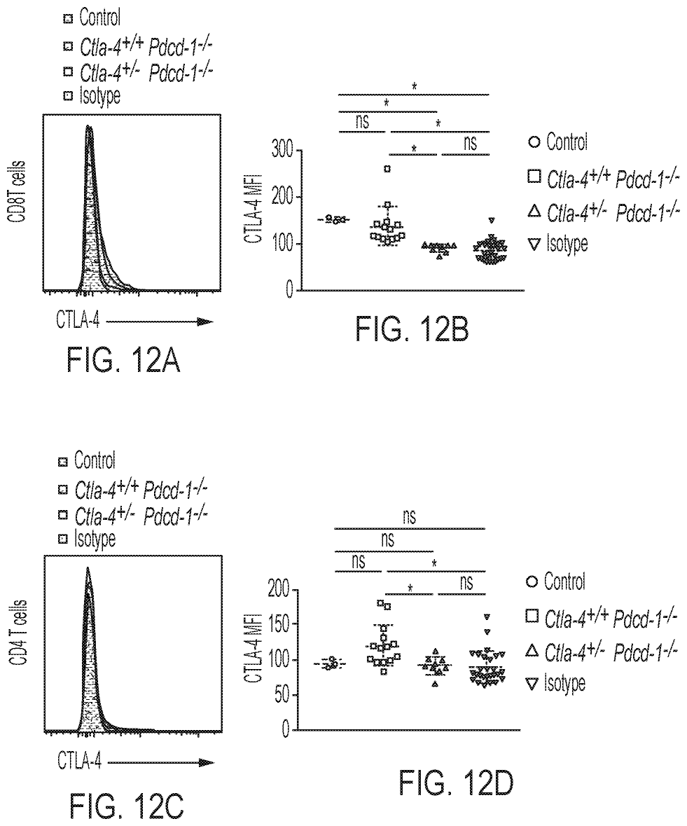

It was next sought to understand the cause of death of Ctla4+/− Pdcd1−/− mice and investigate whether specific tissues were affected. In addition, it was sought to understand whether particular cell types mediated disease etiology. To address these questions, 42 tissues from Ctla4^{+/+} Pdcd1^{-/-} and Ctla4^{+/-} Pdcd1^{-/-} mice were histologically analyzed (see Materials and Methods). Inflammation was observed in multiple tissues including heart, pancreas, lung, liver, and gastrointestional tract. Specific pathologic observations include lymphocytic myocarditis, endarteritis, pulmonary vasculitis, and lymphocytic pancreatitis. Of the most dramatic histological findings, significant immune infiltrate was observed in heart and pancreatic tissues of Ctla4+/− Pdcd1−/− mice (FIG. 3A-D; FIG. 10 and FIG. 11A). Additional pathologic observations more minor in nature, but also notable, include lymphoid infiltrates in the salivary gland, lacrimal gland, harderian gland, and kidney. Other minor observations include hepatic degeneration/necrosis, lymphocytic gastritis, and synovitis although the degree to which these findings associate with genotype remain not fully determined. Interestingly, the more significant histological findings were observed in non-lymphoid peripheral tissues rather than lymphoid organs such as the spleen or lymph nodes. This suggests that peripheral immunological tolerance is specifically breached in Ctla4^{+/-} Pdcd1^{-/-} mice.

To better interrogate nature of the myocardial infiltrates, detailed H&E histological analyses of a larger cohort of mice as well as immunohistochemical staining of similar tissue samples for T cells (utilizing CD3 as a pan-T cell marker) was performed. Myocarditis consisted of significant T cell infiltration in Ctla4^{+/-} Pdcd1^{-/-} mice. Histological analyses also were suggestive of infiltration of other immune populations such as macrophages. These data suggest that the myocarditis seen in the Ctla4+/− Pdcd1−/− mice is histologically similar to patients with ICI-associated myocarditis. The results of a range of histological analyses are summarized below in Tables 3-4.

TABLES 3
Summary of histological evaluation of cardiac and pancreatic lesions.

| | Ctla4^{+/-} Pdcd1^{-/-} mice | | Ctla4^{+/+} Pdcd1^{-/-} mice | | | |
|---|---|---|---|---|---|---|
| Heart | | | | | | |
| Number of mice evaluated | 54 | | 59 | | | |
| Percentage male | 48% | | 56% | | | |
| Percentage female | 52% | | 44% | | | |
| Mean age evaluated | 154.5 days | | 164.4 days | | | |
| | Male | Female | Male | | Female | |
| Percentage of mice with | 23% | 39% | 0.09% | | 19% | |
| Lymphohistiocytic infiltrate histologic | | | | | | |
| score >/= 2 at any location | | | | | | |
| Mean lymphohistiocytic infiltrate histology | 2.46 | 3.71 | 0.76 | | 1.23 | |
| score | | | | | | |
| Percentage of mice with CD3+ | 34.6% | 42.9% | 15.2% | | 19.2% | |
| lymphocyte infiltrate histologic score >/= 2 | | | | | | |
| at any location | | | | | | |
| Mean CD3+ lymphocyte infiltrate histology | 3.04 | 5.73 | 1.59 | | 2.36 | |
| score | | | | | | |
| Pancreas | | | | | | |
| Number of mice | 44 | | 42 | | | |
| Percentage male | 48% | | 45% | | | |
| Percentage female | 52% | | 55% | | | |
| Average age | 139.6 days | | 149.4 days | | | |
| Percentage of mice with histologic score | | | | | | |
| sum >/= 2 (Mean histology score) | Male | Female | Male | | Female | |
| Periductal/perivascular lymphohistiocytic | 71% (4.43) | 87% (5.13) | 11% | (0.68) | 30% | (1.13) |
| infiltrate | | | | | | |
| Periductal/perivascular CD3+ | 62% (3.15) | 77% (3.27) | 39% | (1.22) | 43% | (1.52) |
| lymphocytes | | | | | | |
| Exocrine atrophy | 43% (1.57) | 57% (2.13) | 5% | (0.11) | 0% | (0.04) |

TABLE 4
Histological analyses of Ctla4^{+/-} Pdcd1^{-/-} and littermate
Ctla4^{+/+} Pdcd1^{-/-} control mice. Semi-quantitative histologic scores
across a broad range of tissues. Mouse characteristics including age, sex,
symptoms observed, and genotype are denoted. Lesions considered to be
incidental or mouse strain-related are not reported in the table. Distribution of
lesions by genotype, age, sex, with threshold of scores reported. Not all
anatomic structures were available for evaluation in the examined sections.

| | First group of mice on which complete necropsies performed | | | | | | | |
|---|---|---|---|---|---|---|---|---|
| | Ctla4^{+/-} Pdcd1^{-/-} | | | | Ctla4^{+/+} Pdcd1^{-/-} | | | |
| | Age | | | | | | | |
| | <110 Days | | >110 Days | | <110 Days | | >110 Days | |
| Sex | Male | Female | Male | Female | Male | Female | Male | Female |
| Cardiovascular | | | | | | | | |
| Ventricular | 2/3 | 0/2 | 0/0 | 0/1 | 0/4 | 0/2 | 0/0 | 1/1 |
| endocarditis, | | | | | | | | |
| mononuclear, | | | | | | | | |
| score >/= 1 | | | | | | | | |
| Ventricular | 2/3 | 1/2 | 0/0 | 0/1 | 0/4 | 0/2 | 0/0 | 1/1 |
| myocarditis, | | | | | | | | |
| mononuclear, | | | | | | | | |
| score >/= 1 | | | | | | | | |
| Ventricular | 1/3 | 0/2 | 0/0 | 0/1 | 0/4 | 0/2 | 0/0 | 0/1 |
| epicarditis, | | | | | | | | |
| mononuclear, | | | | | | | | |
| score >/= 1 | | | | | | | | |
| Atrial | 2/3 | 0/1 | 0/0 | 0/1 | 0/4 | 2/2 | 0/0 | 0/1 |
| myocarditis, | | | | | | | | |
| score >/= 1 | | | | | | | | |
| Aortic arteritis, | 1/2 | 0/0 | 0/0 | 0/0 | 0/4 | 0/2 | 0/0 | 0/1 |
| score >/= 1 | | | | | | | | |
| Pancreas | | | | | | | | |
| Insulitis, | 1/3 | 0/2 | 0/0 | 0/1 | 0/4 | 0/2 | 0/0 | 0/1 |
| lymphoid or | | | | | | | | |
| mononuclear, | | | | | | | | |
| score >/= 1 | | | | | | | | |
| Exocrine | 1/3 | 2/2 | 0/0 | 1/1 | 0/4 | 1/2 | 0/0 | 0/1 |
| pancreatitis, | | | | | | | | |
| periductal or | | | | | | | | |
| parenchymal, | | | | | | | | |
| lymphoid or | | | | | | | | |
| mononuclear, | | | | | | | | |
| score >/= 1 | | | | | | | | |
| Loss of | 1/3 | 2/2 | 0/0 | 1/1 | 0/4 | 1/2 | 0/0 | 0/1 |
| exocrine | | | | | | | | |
| parenchyma, | | | | | | | | |
| score >/= 2 | | | | | | | | |
| Pulmonary | | | | | | | | |
| Vasculitis and | 2/3 | 0/2 | 0/0 | 0/1 | 0/4 | /2 | 0/0 | 1/1 |
| perivasculitis, | | | | | | | | |
| chronic, | | | | | | | | |
| multifocal to | | | | | | | | |
| coalescing, | | | | | | | | |
| score >/= 2 | | | | | | | | |
| Renal | | | | | | | | |
| Lymphoid or | 1/3 | 0/2 | 0/0 | 0/1 | 3/4 | 0/2 | 0/0 | 1/1 |
| mononuclear | | | | | | | | |
| infiltrate, | | | | | | | | |
| interstitial, | | | | | | | | |
| hilar, or capsular, | | | | | | | | |
| score >/= 2 | | | | | | | | |
| Cortical tubular | 0/3 | 0/2 | 0/0 | 0/1 | 1/4 | 1/2 | 0/0 | 0/1 |
| basophilia, | | | | | | | | |
| consistent with | | | | | | | | |
| chronic | | | | | | | | |
| progressive | | | | | | | | |
| nephropathy, | | | | | | | | |
| score >/= 2 | | | | | | | | |
| Lymphoid Organs | | | | | | | | |
| Cyst, thymus | 0/3 | 0/0 | 0/0 | 0/1 | 2/4 | 0/2 | 0/0 | 1/1 |
| Hepatic | | | | | | | | |
| Portal | 1/3 | 2/2 | 0/0 | 0/1 | 2/4 | 1/2 | 0/0 | 1/1 |
| mononuclear | | | | | | | | |
| infiltrate, | | | | | | | | |
| scores >/= 1 | | | | | | | | |
| Cytoplasmic | 0/3 | 1/2 | 0/0 | 1/1 | 0/4 | 1/2 | 0/0 | 0/1 |
| alteration, | | | | | | | | |
| eosinophilic, | | | | | | | | |
| hepatocytes, | | | | | | | | |
| scores >/= 2 | | | | | | | | |
| Hepatitis, | 2/3 | 0/2 | 0/0 | 0/1 | 0/4 | 2/2 | 0/0 | 1/1 |
| subacute, focal or | | | | | | | | |
| multifocal, | | | | | | | | |
| scores >/= 2 | | | | | | | | |

Severe atrophy of adipose tissue associated with a wide range of anatomical sites including lung and subcutaneous (skin) was also observed (FIG. 10). It remains unclear whether this phenotype is secondary to the loss of pancreatic exocrine function or a direct effect due to autoimmune recognition of adipose tissue. In addition, atrophy of female reproductive organs was also observed.

These findings are further notable given that the C57BL6/J inbred strain of mice is highly resistant to the development of autoimmunity. For example, consistent with the findings here, mice deficient for PD-1 develop more severe autoimmunity on a Balb/c background compared to a C57BL6/J background (Nishimura et al., 1999; Nishimura et al., 2001). It is also important to note that the autoimmunity that develops due to genetic loss of PD-1 in aged mice is primarily mediated by auto-antibodies. However, antibody levels were not elevated in Ctla4^{+/-} Pdcd1^{-/-} mice (FIG. 11B). This contrasts the lymphocytic infiltration of peripheral tissues observed in Ctla4^{+/-} Pdcd1^{-/-} mice.

Of note, these findings bear striking resemblance to the autoimmunity and other immune related adverse events (irAEs) associated with combination anti-CTLA4 plus anti-PD-1 immune checkpoint blockade therapy (e.g., ipilimumab plus nivolumab) (Sznol et al., 2017). In particular, the severe autoimmunity observed in pancreatic and cardiac tissue observed in Ctla4^{+/-} Pdcd1^{-/-} mice appears to be analogous to the fulminant myocarditis and insulin-dependent diabetes mellitus that are rare but very serious adverse events associated with therapeutic blockade of CTLA-4 and PD-1 (Barroso-Sousa et al., 2018; Johnson et al., 2016; Moslehi et al., 2018). Supportive of the notion that Ctla4^{+/-} Pdcd1^{-/-} mice closely recapitulates this biology, therapy associated myocarditis and diabetes appear to be directly T cell mediated.

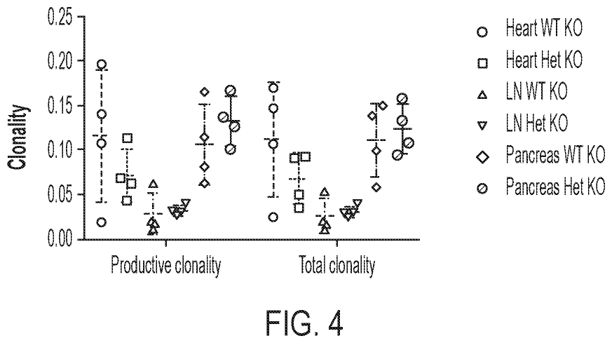

It was then sought to gain insight into the antigen specificity of the T cells that underlies the systemic autoimmunity induced by conditional haploinsufficiency of Ctla4, and in particular, whether particular tissue antigens were being recurrently recognized. To explore this possibility, TCR sequencing was performed on lymph node, heart, and pancreatic tissues from Ctla4^{+/+} Pdcd1^{-/-} and Ctla4^{+/-} Pdcd1^{-/-} mice (see Materials and Methods). Interestingly, no significant changes in T cell clonality were observed between Ctla4^{+/+} Pdcd1^{-/-} and Ctla4^{+/-} Pdcd1^{-/-} mice (FIG. 4). T cell clonality was increased in both strains compared to wild-type or Ctla4^{+/-} mice previously characterized. This suggests that loss of PD-1 is sufficient to induce T cell proliferation and expansion of clonotypes, but the pathogenic activity of these clones is limited by additional mechanisms. This is consistent with the absence or long-latency of autoimmune phenotypes in Pdcd1^{-/-} mice. However, in this context, single-copy loss of Ctla4 appears sufficient to remove additional regulatory constraints, which allows for the manifestation of pathogenic activity by expanded T cell clones.

To assess whether autoimmunity due to conditional haploinsufficiency of Ctla4 in the absence of PD-1 arises solely due to defects in peripheral tolerance, or also defects in central tolerance, thymic development in Ctla4^{+/-} Pdcd1^{-/-} and littermate Ctla4^{+/+} Pdcd1^{-/-} mice were characterized. Mono-allelic loss of Ctla4 did not affect thymocyte composition, consistent with prior reports that CTLA-4 does not play a critical role during thymic development (Chambers et al., 1997; Wei et al., 2019). Consistent with these observations in lymph node derived T cells, thymic-derived Tregs (newly generated and recirculating) derived from Ctla4^{+/-} Pdcd1^{-/-} mice expressed decreased CTLA-4 protein. Together these data indicate that Ctla4 haploinsuffiency leads to a defect in peripheral tolerance rather than a defect in central tolerance.

Finally, the molecular basis of the genetic interaction between Ctla4 and Pdcd1 was investigated. Single copy loss of Ctla4 was hypothesized to lead to subtle changes in signaling and transcriptional outputs that in the context of an otherwise wild-type condition, do not modulate T cell activity or phenotype due to robust buffering within T cell activation signaling pathways. However, in the additional absence of PD-1, or perhaps other negative costimulatory molecules, subtle molecular defects due to Ctla4 haploinsufficiency can manifest overtly. To explore this possibility, we utilized reverse phase proteomic analysis (RPPA) to probe the expression of 238 protein targets in lymph node tissue derived from wild-type, heterozygous, and homozygous Ctla4 knockout mice. This RPPA panel included an array of signaling molecules and phosphorylated epitopes, and thus is well suited to detect changes in canonical signaling pathways.

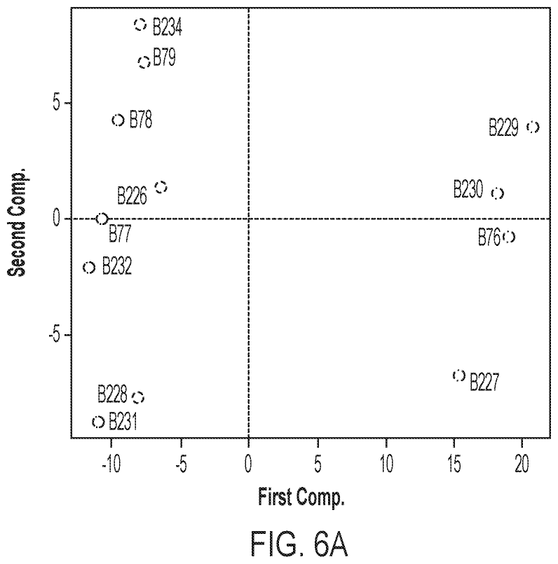

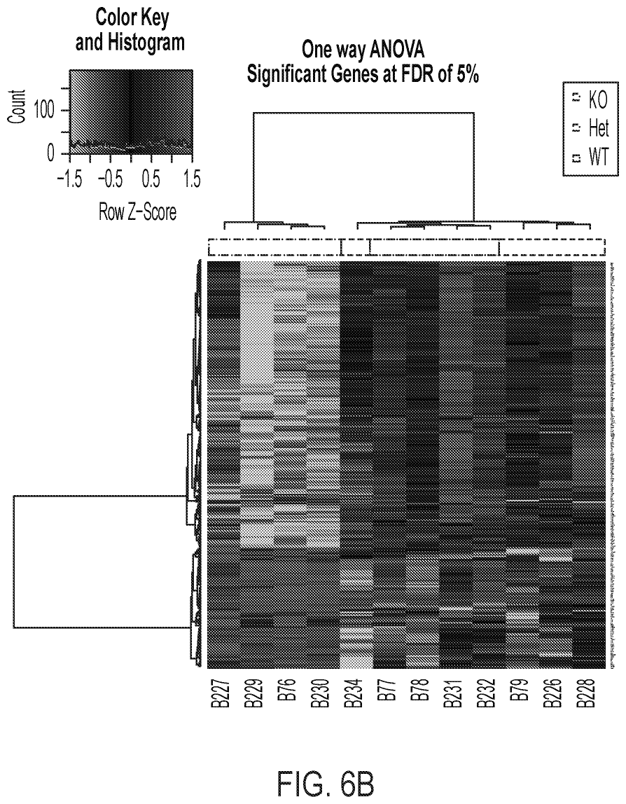

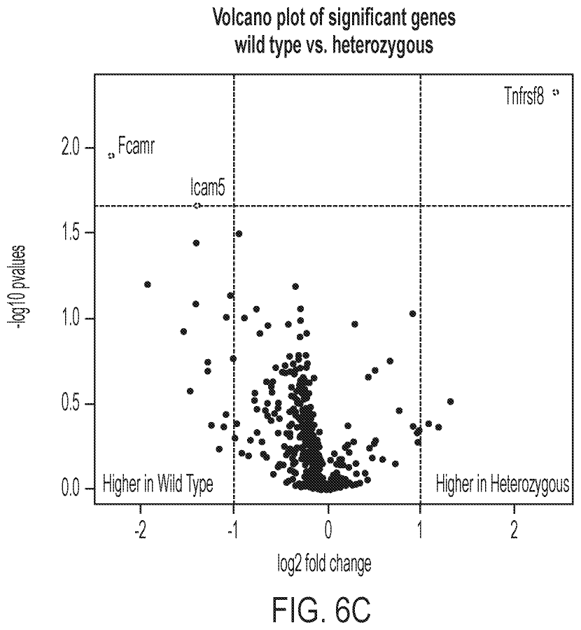

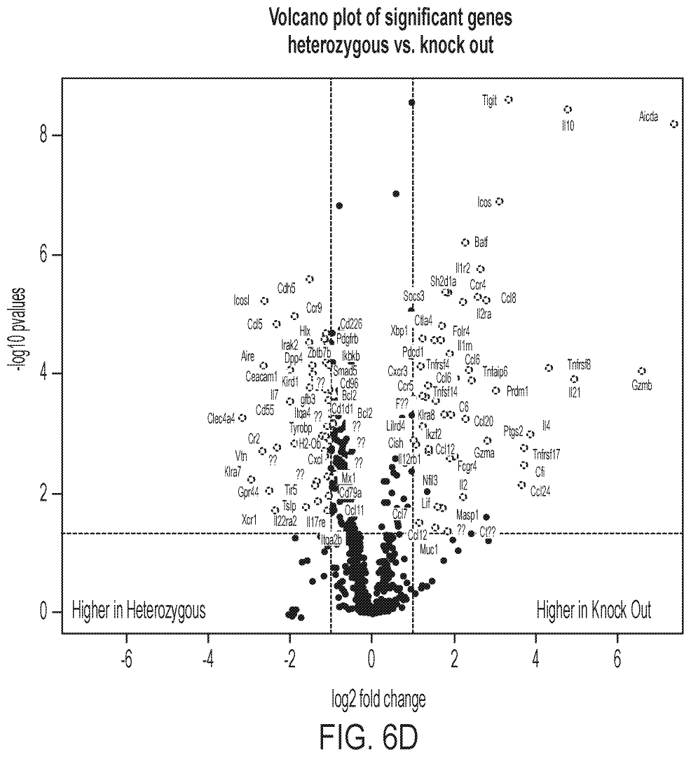

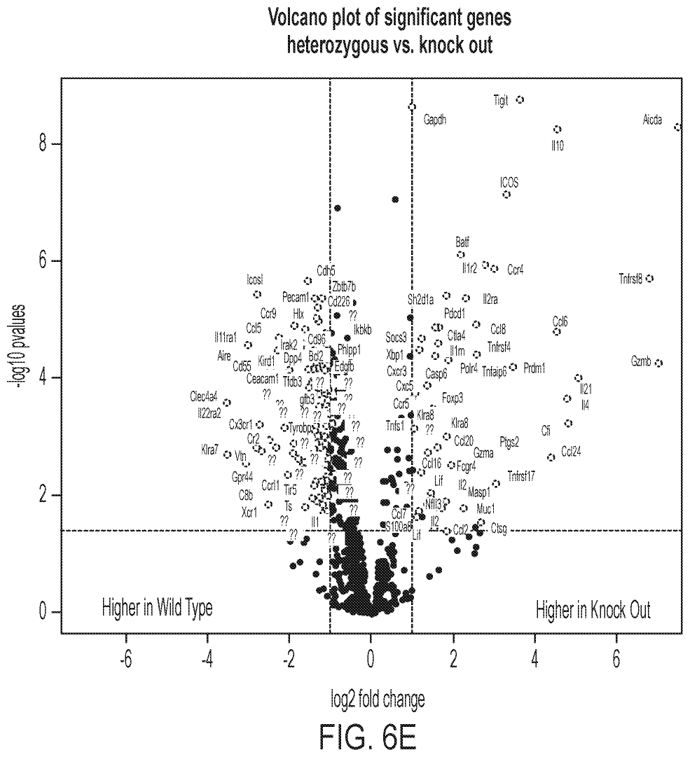

As expected, proteins associated with proliferation pathways were highly upregulated in Ctla4^{-/-} mice compared to littermate controls, consistent with the lymphoproliferative phenotype of Ctla4 knockout mice. This included significant increases in CDK1, p-Rb, p-S6, p-CHK1, and p-STAT3, accompanied by down-regulation of p21 (FIG. 5F). Most notably, principal component analysis (PCA) and unsupervised hierarchical clustering suggest that the proteomic profiles of Ctla4^{+/-} and Ctla4^{+/+} mice are distinct. The difference between these groups is largely driven by the down-regulation of multiple proteins in Ctla4^{+/-} mice, such as p21, DUSP4, and B7-H3. Likewise, gene expression analyses detected differences between Ctla4^{+/-} and Ctla4^{+/+} mice, albeit to a lesser degree, as well as dramatic transcriptional changes in Ctla4^{-/-} mice. Although the observed differences in proteomic and transcriptional profiles may reflect changes in cell intrinsic signaling as well as changes in relative cellular composition given that whole lymph node tissue was analyzed, these findings nonetheless indicate that single copy loss of Ctla4 leads to a subtle molecular haploinsufficiency phenotype. These subtle changes at the proteomic level (FIG. 5) are appear insufficient to modulate immunological responses however, consistent with the absence of such differences at the transcriptional level (FIG. 6). Together these data indicate that PD-1 negative costimulation is sufficient to functionally buffer mono-allelic loss of Ctla4 in mice. This functional buffer is lost in Ctla4^{+/-} Pdcd1^{-/-} mice, which thus allows the development and onset of spontaneous autoimmunity.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barroso-Sousa, R., Barry, W. T., Garrido-Castro, A. C., Hodi, F. S., MM, L., Krop, I. E., and Tolaney, S. M. (2018). Incidence of Endocrine Dysfunction Following the Use of Different Immune Checkpoint Inhibitor Regimens: A Systematic Review and Meta-analysis. JAMA Oncol 4, 173-182.
Besnard, C., Levy, E., Aladjidi, N., Stolzenberg, M. C., Magerus-Chatinet, A., Alibeu, O., Nitschke, P., Blanche, S., Hermine, O., Jeziorski, E., et al. (2018). Pediatric-onset Evans syndrome: Heterogeneous presentation and high frequency of monogenic disorders including LRBA and CTLA4 mutations. Clin Immunol.
Bogue, M. A., Grubb, S. C., Walton, D. O., Philip, V. M., Kolishovski, G., Stearns, T., Dunn, M. H., Skelly, D. A., Kadakkuzha, B., TeHennepe, G., et al. (2018). Mouse Phenome Database: an integrative database and analysis suite for curated empirical phenotype data from laboratory mice. Nucleic Acids Res 46, D843-D850.
Chambers, C. A., Cado, D., Truong, T., and Allison, J. P. (1997). Thymocyte development is normal in CTLA4-deficient mice. Proceedings of the National Academy of Sciences of the United States of America 94, 9296-9301.
Chemnitz, J. M., Parry, R. V., Nichols, K. E., June, C. H., and Riley, J. L. (2004). SHP-1 and SHP-2 associate with immunoreceptor tyrosine-based switch motif of programmed death 1 upon primary human T cell stimulation, but only receptor ligation prevents T cell activation. Journal of immunology 173, 945-954.
Curran, M. A., Montalvo, W., Yagita, H., and Allison, J. P. (2010). PD-1 and CTLA4 combination blockade expands infiltrating T cells and reduces regulatory T and myeloid cells within B16 melanoma tumors. Proceedings of the National Academy of Sciences of the United States of America 107, 4275-4280.
Das, R., Verma, R., Sznol, M., Boddupalli, C. S., Gettinger, S. N., Kluger, H., Callahan, M., Wolchok, J. D., Halaban, R., Dhodapkar, M. V., and Dhodapkar, K. M. (2015). Combination therapy with anti-CTLA4 and anti-PD-1 leads to distinct immunologic changes in vivo. Journal of immunology 194, 950-959.
Havran, W. L., and Allison, J. P. (1988). Developmentally ordered appearance of thymocytes expressing different T-cell antigen receptors. Nature 335, 443-445.
Hou, T. Z., Verma, N., Wanders, J., Kennedy, A., Soskic, B., Janman, D., Halliday, N., Rowshanravan, B., Worth, A., Qasim, W., et al. (2017). Identifying functional defects in patients with immune dysregulation due to LRBA and CTLA4 mutations. Blood 129, 1458-1468.
Hui, E., Cheung, J., Zhu, J., Su, X., Taylor, M. J., Wallweber, H. A., Sasmal, D. K., Huang, J., Kim, J M, Mellman, I., and Vale, R. D. (2017). T cell costimulatory receptor CD28 is a primary target for PD-1-mediated inhibition. Science 355, 1428-1433.
Johnson, D. B., Balko, J. M., Compton, M. L., Chalkias, S., Gorham, J., Xu, Y., Hicks, M., Puzanov, I., Alexander, M. R., Bloomer, T. L., et al. (2016). Fulminant Myocarditis with Combination Immune Checkpoint Blockade. The New England journal of medicine 375, 1749-1755.
Kamphorst, A. O., Wieland, A., Nasti, T., Yang, S., Zhang, R., Barber, D. L., Konieczny, B. T., Daugherty, C. Z., Koenig, L., Yu, K., et al. (2017). Rescue of exhausted CD8 T cells by PD-1-targeted therapies is CD28-dependent. Science 355, 1423-1427.
Keir, M. E., Freeman, G. J., and Sharpe, A. H. (2007). PD-1 regulates self-reactive CD8+ T cell responses to antigen in lymph nodes and tissues. Journal of immunology 179, 5064-5070.
Kroner, A., Mehling, M., Hemmer, B., Rieckmann, P., Toyka, K. V., Maurer, M., and Wiendl, H. (2005). A PD-1 polymorphism is associated with disease progression in multiple sclerosis. Ann Neurol 58, 50-57.
Krummel, M. F., and Allison, J. P. (1996). CTLA4 engagement inhibits IL-2 accumulation and cell cycle progression upon activation of resting T cells. The Journal of experimental medicine 183, 2533-2540.
Kuehn, H. S., Ouyang, W., Lo, B., Deenick, E. K., Niemela, J. E., Avery, D. T., Schickel, J. N.,
Tran, D. Q., Stoddard, J., Zhang, Y., et al. (2014) Immune dysregulation in human subjects with heterozygous germline mutations in CTLA4. Science 345, 1623-1627.
Legoux, F. P., Lim, J. B., Cauley, A. W., Dikiy, S., Ertelt, J., Mariani, T. J., Sparwasser, T., Way, S. S., and Moon, J. J. (2015). CD4+ T Cell Tolerance to Tissue-Restricted Self Antigens Is Mediated by Antigen-Specific Regulatory T Cells Rather Than Deletion Immunity 43, 896-908.
Lek, M., Karczewski, K. J., Minikel, E. V., Samocha, K. E., Banks, E., Fennell, T., O'Donnell-Luria, A. H., Ware, J. S., Hill, A. J., Cummings, B. B., et al. (2016). Analysis of protein-coding genetic variation in 60,706 humans. Nature 536, 285-291.
Moslehi, J. J., Salem, J. E., Sosman, J. A., Lebrun-Vignes, B., and Johnson, D. B. (2018). Increased reporting of fatal immune checkpoint inhibitor-associated myocarditis. Lancet 391, 933.
Nishimura, H., Nose, M., Hiai, H., Minato, N., and Honjo, T. (1999). Development of lupus-like autoimmune diseases by disruption of the PD-1 gene encoding an ITIM motif-carrying immunoreceptor. Immunity 11, 141-151.
Nishimura, H., Okazaki, T., Tanaka, Y., Nakatani, K., Hara, M., Matsumori, A., Sasayama, S., Mizoguchi, A., Hiai, H., Minato, N., and Honjo, T. (2001). Autoimmune dilated cardiomyopathy in PD-1 receptor-deficient mice. Science 291, 319-322.
Parry, R. V., Chemnitz, J. M., Frauwirth, K. A., Lanfranco, A. R., Braunstein, I., Kobayashi, S. V., Linsley, P. S., Thompson, C. B., and Riley, J. L. (2005). CTLA4 and PD-1 receptors inhibit T-cell activation by distinct mechanisms. Molecular and cellular biology 25, 9543-9553.
Postow, M. A., Chesney, J., Pavlick, A. C., Robert, C., Grossmann, K., McDermott, D., Linette, G. P., Meyer, N., Giguere, J. K., Agarwala, S. S., et al. (2015). Nivolumab and ipilimumab versus ipilimumab in untreated melanoma. The New England journal of medicine 372, 2006-2017.
Prokunina, L., Castillejo-Lopez, C., Oberg, F., Gunnarsson, I., Berg, L., Magnusson, V., Brookes, A. J., Tentler, D., Kristjansdottir, H., Grondal, G., et al. (2002). A regulatory polymorphism in PDCD1 is associated with susceptibility to systemic lupus erythematosus in humans. Nature genetics 32, 666-669.
Rau, H., Braun, J., Donner, H., Seissler, J., Siegmund, T., Usadel, K. H., and Badenhoop, K. (2001). The codon 17 polymorphism of the CTLA4 gene in type 2 diabetes mellitus. J Clin Endocrinol Metab 86, 653-655.
Schubert, D., Bode, C., Kenefeck, R., Hou, T. Z., Wing, J. B., Kennedy, A., Bulashevska, A., Petersen, B. S., Schaffer, A. A., Gruning, B. A., et al. (2014). Autosomal dominant immune dysregulation syndrome in humans with CTLA4 mutations. Nature medicine 20, 1410-1416.
Sznol, M., Ferrucci, P. F., Hogg, D., Atkins, M. B., Wolter, P., Guidoboni, M., Lebbe, C., Kirkwood, J. M., Schachter, J., Daniels, G. A., et al. (2017). Pooled Analysis Safety Profile of Nivolumab and Ipilimumab Combination Therapy in Patients With Advanced Melanoma. Journal of clinical oncology: official journal of the American Society of Clinical Oncology 35, 3815-3822.
Tivol, E. A., Borriello, F., Schweitzer, A. N., Lynch, W. P., Bluestone, J. A., and Sharpe, A. H. (1995). Loss of CTLA4 leads to massive lymphoproliferation and fatal multiorgan tissue destruction, revealing a critical negative regulatory role of CTLA4 Immunity 3, 541-547.
Vaidya, B., Imrie, H., Perros, P., Dickinson, J., McCarthy, M. I., Kendall-Taylor, P., and Pearce, S. H. (1999). Cytotoxic T lymphocyte antigen-4 (CTLA4) gene polymorphism confers susceptibility to thyroid associated orbitopathy. Lancet 354, 743-744.
Walunas, T. L., Bakker, C. Y., and Bluestone, J. A. (1996). CTLA4 ligation blocks CD28-dependent T cell activation. The Journal of experimental medicine 183, 2541-2550.
Waterhouse, P., Penninger, J. M., Timms, E., Wakeham, A., Shahinian, A., Lee, K. P., Thompson, C. B., Griesser, H., and Mak, T. W. (1995). Lymphoproliferative disorders with early lethality in mice deficient in Ctla4. Science 270, 985-988.
Wei, S. C., Levine, J. H., Cogdill, A. P., Zhao, Y., Anang, N. A. S., Andrews, M. C., Sharma, P., Wang, J., Wargo, J. A., Pe′er, D., and Allison, J. P. (2017). Distinct Cellular Mechanisms Underlie Anti-CTLA4 and Anti-PD-1 Checkpoint Blockade. Cell 170, 1120-1133 e1117.
Wei, S. C., Duffy, C. R., and Allison, J. P. (2018). Fundamental Mechanisms of Immune Checkpoint Blockade Therapy. Cancer Discovery 8, 1-18.
Wolchok, J. D., Kluger, H., Callahan, M. K., Postow, M. A., Rizvi, N. A., Lesokhin, A. M., Segal,
N. H., Ariyan, C. E., Gordon, R. A., Reed, K., et al. (2013). Nivolumab plus ipilimumab in advanced melanoma. The New England journal of medicine 369, 122-133.
Zalloua, P. A., Abchee, A., Shbaklo, H., Zreik, T. G., Terwedow, H., Halaby, G., and Azar, S. T. (2004). Patients with early onset of type 1 diabetes have significantly higher GG genotype at position 49 of the CTLA4 gene. Hum Immunol 65, 719-724.

1. A mouse whose genome comprises: (i) a heterozygous loss-of-function allele of a Ctla4 gene and (ii) a homozygous loss-of-function allele of a Pdcd1 gene.

2. The mouse of claim 1, wherein the mouse has a C57BL/6J genetic background.

3. The mouse of claim 1, wherein the heterozygous loss-of-function allele of a Ctla4 gene is further defined as a heterozygous insertion of a neomycin resistance cassette into exon 3 of the Ctla4 gene.

4. The mouse of claim 1, wherein the homozygous loss-of-function allele of the Pdcd1 gene is further defined as a homozygous deletion of exons 2 and 3 of the Pdcd1 gene.

5. The mouse of claim 1, wherein the mouse is a Ctla4^{tm1All}Pdcd1^{tm1.1Shr} mouse.

6. The mouse of claim 1, wherein the mouse suffers from autoimmunity.

7. The mouse of claim 6, wherein the autoimmunity is cardiac autoimmunity or pancreatic autoimmunity.

8. The mouse of claim 7, wherein the cardiac autoimmunity is myocarditis.

9. The mouse of claim 8, wherein the myocarditis is fulminant myocarditis.

10. The mouse of claim 7, wherein the pancreatic autoimmunity is insulin-dependent diabetes mellitus.

11. The mouse of claim 7, wherein the pancreatic autoimmunity comprises pancreatic exocrine destruction or pancreatic islet destruction.

12. The mouse of claim 6, wherein the autoimmunity is lymphocytic myocarditis, endarteritis, pancreatic exocrine destruction, pulmonary vasculitis, adipose tissue atrophy, hepatic inflammation, atrophy of female reproductive organs, gastrointestinal tract inflammation, synovitis, or lymphocytic infiltration of the kidney, salivary gland, lacrimal gland, or stomach.

13. The mouse of any one of claims 1-12, wherein the mouse is a female mouse.

14. The mouse of any one of claims 1-12, wherein the mouse is a male mouse.

15. A cell isolated from a mouse of any one of claims 1-14.

16. The cell of claim 15, wherein the cell is an immune cell.

17. The cell of claim 15, wherein the cell is a T cell.

18. A method for screening at least one candidate agent in the mouse according to any one of claims 1-14, comprising administering one or more candidate agent to the mouse.

19. The method of claim 18, further comprising screening the at least one candidate agent in a mouse comprising (i) a homozygous wild-type Ctla4 gene and (ii) a homozygous loss-of-function allele of a Pdcd1 gene.

20. The method of claim 18, further comprising screening the at least one candidate agent in a mouse comprising (i) a homozygous wild-type Ctla4 gene and (ii) a homozygous wild-type Pdcd1 gene.

21. The method of claim 18, further comprising screening the at least one candidate agent in a mouse comprising (i) a homozygous wild-type Ctla4 gene and (ii) a heterozygous loss-of-function allele of a Pdcd1 gene.

22. The method of claim 18, further comprising screening the at least one candidate agent in a mouse comprising (i) a heterozygous loss-of-function allele of a Ctla4 gene and (ii) a homozygous wild-type Pdcd1 gene.

23. The method of claim 18, further comprising screening the at least one candidate agent in a mouse comprising (i) a heterozygous loss-of-function allele of a Ctla4 gene and (ii) a heterozygous loss-of-function of a Pdcd1 gene.

24. The method of any one of claims 18-23, wherein the at least one candidate agent is screened for its ability to accelerate the development of an immune-related adverse event or immune-related condition.

25. The method of any one of claims 18-23, wherein the at least one candidate agent is screened for its ability to worsen the severity of an immune-related adverse event or immune-related condition.

26. The method of any one of claims 18-23, wherein the at least one candidate agent is screened for its ability to increase the penetrance of an immune-related adverse event or immune-related condition in a population of the mice.

27. The method of any one of claims 18-23, wherein the at least one candidate agent is screened for its ability to mitigate an immune-related adverse event or immune-related condition.

28. The method of claim 27, wherein mitigating an immune-related adverse event or immune-related condition is further defined as preventing the development of the immune-related adverse event or immune-related condition.

29. The method of claim 27, wherein mitigating an immune-related adverse event or immune-related condition is further defined as decreasing the severity of the immune-related adverse event or immune-related condition.

30. The method of any one of claims 18-23, wherein the at least one candidate agent is screened for efficacy.

31. The method of any one of claims 18-30, wherein the candidate agent is an anti-cancer therapy.

32. The method of any one of claims 18-30, wherein the candidate agent is a pathogen, stress, an injury, and/or a diet.

33. The method of any one of claims 18-30, wherein the candidate agent is a syngeneic tumor cell.

34. The method of any one of claims 18-30, wherein the candidate agent is a CTLA-4 immunoglobulin fusion protein, a steroid, an agent that depletes a specific population of immune cells, a cytokine modulating agent, or an immunosuppressive agent.

35. The method of any one of claims 24-29, wherein the immune-related adverse event is an autoimmunity.

36. The method of any one of claims 24-29, wherein the immune-related condition is an autoimmune condition.

37. The method of any one of claims 24-29, wherein the immune-related adverse event is acute.

38. The method of any one of claims 24-29, wherein the immune-related adverse event is chronic.

39. The method of any one of claims 24-29, wherein the immune related condition is chronic.

40. The method of any one of claims 24-29, wherein the immune related condition is acute.

41. The method of any one of claims 24-29, wherein the immune-related adverse event or immune related condition is inflammation.

42. The method of claim 41, wherein the inflammation is acute or chronic.

43. The method of any one of claims 24-29, wherein the immune-related adverse event or immune-related condition is an autoimmunity that represents an autoimmunity induced by a checkpoint blockade therapy in humans or represents an immune-related adverse event in humans.

44. The method of any one of claims 24-29, wherein the immune-related adverse event or immune-related condition is cardiac autoimmunity or pancreatic autoimmunity.

45. The method of claim 44, wherein the cardiac autoimmunity is myocarditis.

46. The method of claim 45, wherein the myocarditis is fulminant myocarditis.

47. The method of claim 44, wherein the pancreatic autoimmunity is insulin-dependent diabetes mellitus.

48. The mouse of claim 44, wherein the pancreatic autoimmunity comprises pancreatic exocrine destruction or pancreatic islet destruction.

49. The mouse of any one of claims 24-29, wherein the immune-related adverse event or immune-related condition is lymphocytic myocarditis, endarteritis, pancreatic exocrine destruction, pulmonary vasculitis, adipose tissue atrophy, hepatic inflammation, atrophy of female reproductive organs, gastrointestinal tract inflammation, synovitis, or lymphocytic infiltration of the kidney, salivary gland, lacrimal gland, or stomach.
